(19) United States
(12) Patent Application Publication
(10) Pub. No.: US 2024/0196906 A1
(43) Pub. Date: Jun. 20, 2024

(54) PESTICIDAL MINICELLS AND COMPOSITIONS THEREOF FOR AGRICULTURAL APPLICATIONS

(71) Applicant: Invaio Sciences, Inc., Cambridge, MA (US)

(72) Inventors: Maier Steve AVENDAÑO AMADO, Chelsea, MA (US); Rama Krishna SIMHADRI, Natick, MA (US); Duane Lee KRISTENSEN, Somerville, MA (US); James Aaron KRAEMER, Cambridge, MA (US)

(73) Assignee: Invaio Sciences, Inc., Cambridge, MA (US)

(21) Appl. No.: 18/555,429

(22) PCT Filed: Apr. 14, 2022
(86) PCT No.: PCT/US2022/024821

§ 371 Date: Oct. 13, 2023

Related U.S. Application Data
(60) Provisional application No. 63/175,488, filed on Apr. 15, 2021.

(51) Int. Cl. A01N 63/22; A01N 25/02; C07K 14/325; C12N 1/20; C12R 1/125; C12R 1/19

(52) U.S. Cl. CPC A01N 63/22; A01N 25/02; C07K 14/325; C12N 1/20; C12R 2001/125; C12R 2001/19

(57) ABSTRACT

This disclosure provides pesticidal minicells, compositions including pesticidal minicells, and methods of making pesticidal minicells.

### CROSS-REFERENCE TO RELATED APPLICATIONS

This application claims priority to U.S. Provisional Patent Application No. 63/175,488 filed Apr. 15, 2021, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165852001840SEQLIST.TXT, date recorded: Apr. 13, 2022, size: 1,410 bytes).

### FIELD

The present disclosure relates to pesticidal minicells, compositions including pesticidal minicells, and methods of making pesticidal minicells.

### BACKGROUND

A need exists for delivery vectors capable of targeting cells and delivering biological agents, compositions containing such delivery vectors, and associated methods of delivering said vectors to cells, thereby modulating biological systems including animal, plant, and insect cells, tissues, and organisms. In particular, a need exists for delivery vectors that function both as delivery vectors and an active ingredient (e.g., a pesticidal active ingredient).

### BRIEF SUMMARY

Accordingly, the present disclosure provides compositions including pesticidal minicells. Pesticidal minicells are produced from pesticidal parent bacteria, which can suppress pests including insects, fungi, and nematodes. Pesticidal minicells retain the pesticidal activity of the parent cells and are naturally degradable. Further, pesticidal minicells can be used to produce, amplify, and deliver a variety of biological active ingredients, including protein toxins and nucleic acids. The present disclosure further provides methods of producing pesticidal minicells by modifying the cell partitioning function of the pesticidal parent bacteria.

An aspect of the disclosure includes a pesticidal composition including a liquid carrier phase; and a plurality of pesticidal minicells dispersed in the carrier phase, wherein the plurality of pesticidal minicells are derived from a plurality of a pesticidal parent bacterium including at least one genetic mutation causing a modification in a cell partitioning function of the parent bacterium, and wherein the plurality of pesticidal minicells are present at a particle concentration sufficient to control at least one pest in or on a plant when the composition is applied to the plant. In some embodiments of this aspect, control includes at least one of: a reduction in pest number on the plant when compared to a check plant not treated with the composition, and a reduction in physical damage to the plant when compared to a check plant not treated with the composition. In further embodiments of this aspect, the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, at least 20% reduction, an at least 25% reduction, an at least 30% reduction, an at least 40% reduction, an at least 50% reduction, an at least 60% reduction, an at least 70% reduction, or an at least 80% reduction. In other embodiments of this aspect, which may be combined with any of the preceding embodiments, at least a portion of the plurality of pesticidal minicells further include at least one of: an exogenous pesticidal protein toxin, an exogenous pesticidal nucleic acid, and an exogenous pesticidal active ingredient. In some embodiments of this aspect, the portion of the plurality of pesticidal minicells further include an exogenous expression cassette coded to express either or both of the exogenous pesticidal protein toxin and the exogenous pesticidal nucleic acid. In some embodiments of this aspect, the exogenous pesticidal protein toxin includes at least one of a Pir toxin or a Cry toxin. In some embodiments of this aspect, the exogenous pesticidal nucleic acid is a double-stranded RNA (dsRNA) or a hairpin RNA (hpRNA). In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity.

In some embodiments of this aspect, which may be combined with any of the preceding embodiments, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC poly peptide, a minD polypeptide, or a minE poly peptide; wherein the cell division topological specificity factor is selected from the group of a minE polypeptide or a DivIVA polypeptide, and wherein the septum machinery component is selected from the group of a ftsZ polypeptide or a fisA polypeptide.

In some embodiments of this aspect, which may be combined with any of the preceding embodiments, the pesticidal parent bacterium is selected from the group of Streptomyces avermitilis, Saccharopolyspora spinose, Bacillus thuringiensis, Brevibacillus laterosporus, Clostridium bifermentans. Bacillus popilliae, Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, or Escherichia coli. In further embodiments of this aspect, the pesticidal parent bacterium is selected from the group of Bacillus subtilis strain R11477, Bacillus subtilis strain AHC 6633, Bacillus subtilis strain AHC 21332, Bacillus subtilis strain 168, Bacillus subtilis strain ATVC 9943, Bacillus subtilis strain QST713, and Bacillus subtilis strain NCIB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens LMG 5-29032 Bacillus amyloliquefaciens MBI600, Bacillus amyloliquefaciens CECT8836, or Bacillus amyloliquefaciens M4 (S499). In some embodiments of this aspect, the pesticidal parent bacterium is Photorhabdus luminescens, and wherein the pesticidal minicell includes the exogenous pesticidal protein toxin Pir. In some embodiments of this aspect, the pesticidal parent bacterium is Bacillus subtilis, and wherein the pesticidal minicell includes the exogenous pesticidal molecule. In some embodiments of this aspect, the pesticidal parent bacterium is a genetically modified Escherichia coli expressing one or more exogenous pesticidal active ingredients.

In some embodiments of this aspect, which may be combined with any of the preceding embodiments, the composition is applied to the plant as at least one of a foliar treatment, an injection treatment, a pre-emergence treatment, and a post-emergence treatment. In other embodiments of this aspect, which may be combined with any of the preceding embodiments, the composition is formulated as at least one of a Ready To Use (RTU) formulation, a suspension concentrate, a tank-mix, an aerosol, a seed treatment, a root dip, a soil treatment, an irrigation formulation, a sprinkler formulation, and a drench treatment. In some embodiments of this aspect, the composition is formulated as the seed treatment. In further embodiments of this aspect, the composition is applied at a rate of about 1×10² to about 1×10⁹ particle/seed, and wherein the rate is determined based on seed size. In further embodiments of this aspect, the composition is applied at a rate of about 1×10⁴ particle/seed. In other embodiments of this aspect, the composition is formulated as the root dip. In further embodiments of this aspect, the composition is applied at a rate of about 1×10³ to about 1×10⁸ particle/plant root system. Further embodiments of this aspect, which may be combined with any of the preceding embodiments, further include agrochemical surfactants, wherein the agrochemical surfactants improve at least one of the characteristics of sprayability, spreadability, and injectability. In further embodiments of this aspect, the liquid carrier phase is aqueous or oil.

Other embodiments of this aspect, which may be combined with any of the preceding embodiments, further include at least one of: an exogenous pesticidal protein toxin, an exogenous pesticidal nucleic acid, and an exogenous pesticidal active ingredient dispersed in the carrier phase. In some embodiments of this aspect, the exogenous pesticidal protein toxin includes a Pir toxin or a Cry toxin. In some embodiments of this aspect, the exogenous pesticidal nucleic acid is a double-stranded RNA (dsRNA) or precursor thereof, a hairpin RNA (hpRNA) or precursor thereof, or a microRNA (miRNA) or precursor thereof. In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity. In further embodiments of this aspect, the composition is formulated as the seed treatment. In some embodiments of this aspect, the exogenous pesticidal protein toxin, the exogenous pesticidal nucleic acid, or the exogenous pesticidal active ingredient dispersed in the carrier phase is present in an amount from about 1 g to about 10 g per 100 kg of seed. In some embodiments of this aspect, the exogenous pesticidal protein toxin, the exogenous pesticidal nucleic acid, or the exogenous pesticidal active ingredient dispersed in the carrier phase is present in an amount of about 1×10⁴ particle/seed. In further embodiments of this aspect, the composition is formulated as the root dip. In some embodiments of this aspect, the exogenous pesticidal protein toxin, the exogenous pesticidal nucleic acid, or the exogenous pesticidal active ingredient dispersed in the carrier phase is present from about 25 mg to about 200 mg active ingredient/L. In some embodiments of this aspect, the exogenous pesticidal protein toxin, the exogenous pesticidal nucleic acid, or the exogenous pesticidal active ingredient dispersed in the carrier phase is present in an amount of about 1×10³ to about 1×10³ particle/plant root system. In some embodiments of this aspect, which may be combined with any of the preceding embodiments, the minicell particle concentration is in the range of about 1×10² to about 8×10¹⁴. In some embodiments of this aspect, the pesticidal activity of the minicell and the pesticidal activity of the exogenous pesticidal protein toxin, the exogenous pesticidal nucleic acid, or the exogenous pesticidal active ingredient target the same pest. In other embodiments of this aspect, the pesticidal activity of the minicell and the pesticidal activity of the exogenous pesticidal protein toxin, the exogenous pesticidal nucleic acid, or the exogenous pesticidal active ingredient target different pests. In some embodiments of this aspect that may be combined with any of the preceding embodiments having an exogenous component dispersed in the carrier phase, the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target the same pest. In other embodiments of this aspect that may be combined with any of the preceding embodiments having an exogenous component dispersed in the carrier phase, the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target different pests.

In some embodiments of this aspect, which may be combined with any of the preceding embodiments, the at least one pest is selected from the group of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Collelotrichum spp., Botrylis spp., and Cercospora spp. In some embodiments of this aspect, which may be combined with any of the preceding embodiments, the pesticidal minicell remains stable and retains pesticidal activity for at least 8 months, at least 9 months, at least 10 months, at least 11 months, at least 12 months, or at least 13 months.

A further aspect of the disclosure includes methods of making pesticidal minicells, including the steps of: (a) providing a pesticidal parent bacterium including at least one genetic mutation causing a modification in a cell partitioning function of the parent bacterium; (b) growing the pesticidal parent bacterium under conditions allowing the formation of pesticidal minicells; and (c) purifying pesticidal minicells using centrifugation, tangential flow filtration (TFF), or TFF and centrifugation. In some embodiments of this aspect, step (c) produces about 10¹⁰ pesticidal minicells per liter, about 10¹¹ pesticidal minicells per liter, about 10¹² pesticidal minicells per liter, about 10¹³ pesticidal minicells per liter, about 10¹⁴ pesticidal minicells per liter, about 10¹⁵ pesticidal minicells per liter, about 10¹⁶ pesticidal minicells per liter, or about 10¹⁷ pesticidal minicells per liter. Some embodiments of this aspect, which may be combined with any of the preceding embodiments, further include step (d) drying the pesticidal minicells to produce a shelf-stable pesticidal minicell composition. In some embodiments of this aspect, the shelf-stable pesticidal minicell composition retains pesticidal activity for at least 8 months, at least 9 months, at least 10 months, at least 11 months, at least 12 months, or at least 13 months. In some embodiments of this aspect, which may be combined with any of the preceding embodiments, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC polypeptide, a minD polypeptide, or a minE polypeptide; wherein the cell division topological specificity factor is selected from the group of a minE polypeptide or a DivIVA polypeptide, and wherein the septum machinery component is selected from the group of a ftsZ polypeptide or a ftsA polypeptide.

An additional aspect of the disclosure includes a pesticidal minicell-producing parent bacterium, wherein (i) the pesticidal parent bacterium includes a genetic mutation that modifies a cell partitioning function of the parent bacterium; and (ii) the pesticidal parent bacterium exhibits a commercially relevant pesticidal activity with an LD50 against at least one plant pest of less than 100 mg/kg. In other embodiments of this aspect, modifying the cell partitioning function of the parent bacterium includes modifying the level or activity of at least one of a z-ring inhibition protein, a cell division topological specificity factor, or a septum machinery component. In some embodiments of this aspect, which may be combined with any of the preceding embodiments, the z-ring inhibition protein is selected from the group of a minC polypeptide, a minD poly peptide, or a minE poly peptide; wherein the cell division topological specificity factor is selected from the group of a minE polypeptide or a DivIVA polypeptide, and wherein the septum machinery component is selected from the group of a ftsZ poly peptide or a ftsA polypeptide.

Yet another aspect of the disclosure includes methods of controlling a pest, the method including: applying the pesticidal composition of any one of the preceding embodiments to a plant or an area to be planted. In some embodiments of this aspect, the applying includes at least one of an injection application, a foliar application, a pre-emergence application, or a post-emergence application. In some embodiments of this aspect, which may be combined with any of the preceding embodiments, control includes at least one of: a reduction in pest number on the plant when compared to a check plant not treated with the composition, and a reduction in physical damage to the plant when compared to a check plant not treated with the composition. In further embodiments of this aspect, the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction. In other embodiments of this aspect, which may be combined with any of the preceding embodiments, the composition is formulated as at least one of a Ready To Use (RTU) formulation, a suspension concentrate, a tank-mix, an aerosol, a seed treatment, a root dip, a soil treatment, an irrigation formulation, a sprinkler formulation, or a drench treatment. In some embodiments of this aspect, which may be combined with any of the preceding embodiments, the pest is selected from the group of Diamondback moth (DBM). Red flour beetle (RFB), Colorado potato beetle (CPB), Mediterranean flour moth, Fall armyworm (FAW). Asian spotted bollworm, Lepidoptera spp., Coleoptera spp., Diptera spp., Phytophthora spp., Armillaria spp., Colletotrichium spp., Botrytis spp., or Cercospora spp.

Still another aspect of the disclosure includes a wettable powder including: a plurality of dried pesticidal minicells derived from a plurality of a pesticidal parent bacterium including at least one genetic mutation causing a modification in a cell partitioning function of the parent bacterium, wherein the wettable powder is configured to be dispersed in an aqueous carrier to create a pesticidal composition for controlling at least one pest in or on a plant when the composition is applied to the plant. Some embodiments of this aspect further include an agrochemically acceptable solid carrier component including at least one of: a clay component, a kaolin component, a talc component, a chalk component, a calcite component, a quartz component, a pumice component, a diatomaceous earth component, a vermiculite component, a silicate component, a silicon dioxide component, a silica powder component, an aluminum component, an ammonium sulfate component, an ammonium phosphate component, a calcium carbonate component, an urea component, a sugar component, a starch component, a sawdust component, a ground coconut shell component, a ground corn cob component, and a ground tobacco stalk component. In some embodiments of this aspect, which may be combined with any of the preceding embodiments, the aqueous carrier includes water. In some embodiments of this aspect, which may be combined with any of the preceding embodiments, control includes at least one of: a reduction in pest number on the plant when compared to a check plant not treated with the composition, and a reduction in physical damage to the plant when compared to a check plant not treated with the composition. In a further embodiment of this aspect, the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction. In some embodiments of this aspect, which may be combined with any of the preceding embodiments, the at least one pest is selected from the group of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., or Cercospora spp. In other embodiments of this aspect, which may be combined with any of the preceding embodiments, modifying the cell partitioning function of the parent bacterium includes modifying the level or activity of at least one of a z-ring inhibition protein, a cell division topological specificity factor, or a septum machinery component. In further embodiments of this aspect, the z-ring inhibition protein is selected from the group of a minC polypeptide, a minD polypeptide, or a minE polypeptide; wherein the cell division topological specificity factor is selected from the group of a minE polypeptide or a DivIVA polypeptide, and wherein the septum machinery component is selected from the group of a ftsZ polypeptide or a ftsA polypeptide.

A further aspect of the disclosure includes a plantable composition including: a seed; and a coating covering the seed, wherein the coating includes a plurality of pesticidal minicells derived from a plurality of a pesticidal parent bacterium including at least one mutation causing a modification in a cell partitioning function of the parent bacterium, and wherein the plurality of pesticidal minicells are present at a particle concentration sufficient to result in pesticidal activity on at least one pest feeding on the seed or a seedling emerging therefrom. In some embodiments of this aspect, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC polypeptide, a minD polypeptide, or a minE polypeptide; wherein the cell division topological specificity factor is selected from the group of a minE poly peptide or a DivIVA polypeptide, and wherein the septum machinery component is selected from the group of a ftsZ polypeptide or a ftsA polypeptide. In some embodiments of this aspect, which may be combined with any of the preceding embodiments, the pesticidal parent bacterium is selected from the group of Streptomyces amermitilis, Saccharopolyspora spinose, Bacillus thuringiensis, Brevibacillus laterosporus, Clostridium bifermentans, Bacillus popilliae, Bacillus subtilis, Bacillus amvlohquefaciens, Photorhabdus luminescens, Xenorhabdus nematophila, Serratia entomophila, Yersinia entomophaga, Pseudomonas entomophila, Burkholderia spp., Chromobacterium subtsugae, or Escherichia coli. In further embodiments of this aspect, the pesticidal parent bacterium is selected from the group of Bacillus subtilis strain RTI477, Bacillus subtilis strain AFTCC 6633, Bacillus subtilis strain ATCC 21332, Bacillus subtilis strain 168, Bacillus subtilis strain ACC 9943, Bacillus subtilis strain QST713, and Bacillus subtilis strain NCIB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens LMG 5-29032, Bacillus amyloliquefaciens MBI600, Bacillus amyloliquefaciens CECT8836, or Bacillus amyloliquefaciens M4 (S499). In some embodiments of this aspect, which may be combined with any of the preceding embodiments, the coating includes a particle concentration of about 1×10² to about 1×10⁹ particle/seed, and wherein the concentration is determined based on seed size. In further embodiments of this aspect, the particle concentration includes about 1×10⁴ particle/seed.). In some embodiments of this aspect, which may be combined with any of the preceding embodiments, the at least one pest is selected from the group of Diamondback moth (DBM). Red flour beetle (RFB), Colorado potato beetle (CPB). Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., or Cercospora spp. In some embodiments of this aspect, which may be combined with any of the preceding embodiments, the seed is from a plant selected from the group of soybean, strawberry, blackcurrant, white currant, redcurrant, blackberry, raspberry, tomato, pepper, chili, potato, eggplant, cucumber, lettuce, chicory, brassicas, corn, wheat, rice, canola, melon, kale, carrot, or bean.

An additional aspect of the disclosure includes a pesticidal composition including: a pesticidal minicell, wherein the pesticidal minicell is derived from a pesticidal parent bacterium including at least one genetic mutation causing a modification in a level or activity of one or more cell partitioning function factors selected from the group of a minC polypeptide, a minD polypeptide, a minE polypeptide, a ftsZ poly peptide, a ftsA polypeptide, a parA polypeptide, a parB polypeptide, a DivIVA polypeptide, or a combination thereof. In some embodiments of this aspect, the pesticidal minicell includes at least one of: an exogenous pesticidal protein toxin, an exogenous pesticidal nucleic acid, and an exogenous pesticidal active ingredient. In some embodiments of this aspect, the pesticidal minicell further includes an exogenous expression cassette coded to express either or both of the exogenous pesticidal protein toxin and the exogenous pesticidal nucleic acid. In some embodiments of this aspect, the exogenous pesticidal protein toxin includes at least one of a Pir toxin or a Cry toxin. In some embodiments of this aspect, the exogenous pesticidal nucleic acid is a double-stranded RNA (dsRNA) or a hairpin RNA (hpRNA). In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity. In some embodiments of this aspect, which may be combined with any of the preceding embodiments, the pesticidal activity of the minicell and the pesticidal activity of the exogenous pesticidal protein toxin, the exogenous pesticidal nucleic acid, or the exogenous pesticidal active ingredient target the same pest. In other embodiments of this aspect, which may be combined with any of the preceding embodiments, the pesticidal activity of the minicell and the pesticidal activity of the exogenous pesticidal protein toxin, the exogenous pesticidal nucleic acid, or the exogenous pesticidal active ingredient target different pests. In some embodiments of this aspect, which may be combined with any of the preceding embodiments, the at least one pest is selected from the group of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phylophthora spp., Armillaria spp., Colletotrichum spp., Botrytis spp., or Cercospora spp. In some embodiments of this aspect, which may be combined with any of the preceding embodiments, the pesticidal minicell remains stable and retains pesticidal activity for at least 8 months, at least 9 months, at least 10 months, at least 11 months, at least 12 months, or at least 13 months.

These and other aspects of the invention are set forth in more detail in the description of the invention below.

### DESCRIPTION OF THE FIGURES

The present application can be understood by reference to the following description taken in conjunction with the accompanying figures.

FIG. 1 depicts a sequencing map showing that the Photorhabdus luminescens ftsZ gene was successfully inserted into the expression vector. Primer oLK015 reads from the left and primer oAF086 reads from the right (primer sequences in Table 1).

FIG. 2 depicts a graph showing the OD₆₀₀ values of P. luminescens in different media tested for growth over a 48-hour time period.

FIGS. 3A-3C show assays characterizing pesticidal minicells produced from P. luminescens. FIG. 3A is a phase contrast microscopy image of a culture of a minicell producing P. luminescens strain before (on left, “Parent cells”) and after minicell isolation (on right, “ADAS particle”). Parent bacterial cells are indicated by arrows on left, while minicells are indicated by arrows on right. FIG. 3B is a graph of particle size distribution and concentration for the P. luminescens strain TT01 (black) and the P. luminescens strain Kleinni (grey) measured by counting with a Spectradyne nCS1. FIG. 3C shows an image of a Western blot for cytosolic chaperone GroEL. The isolated minicells contain GroEL.

FIGS. 4A-4C show the results of LD50 assays in which Plutella xylostella (Diamondback Moth; DBM) were treated with pesticidal compositions containing minicells produced from P. luminescens. FIG. 4A shows the results of an artificial diet LD50 assay in which DBM larvae were fed a series of concentrations of the minicell particles derived from P. luminescens strain TT01. FIG. 4B shows the results of an artificial diet LD50 assay in which DBM larvae were fed a series of concentrations of the minicell particles derived from P. luminescens strain Kleinni. In FIGS. 4A-4B, mortality was recorded 3 days after feeding. FIG. 4C shows the results of a leaf disk assay LD50 assay in which DBM larvae were fed a series of concentrations of the minicell particles derived from P. luminescens strains TT01 or Kleinii and mortality was recorded 3 days later.

FIGS. 5A-5B show the results of insect mortality assays comparing the effects of minicells produced from P. luminescens on Diamondback Moth (DBM), Fall Army Worm (FAW), Beet Army Worm (BAW), and European Corn Borer (ECB). FIG. 5A shows mortality assays with minicells derived from P. luminescens strain TT01. FIG. 5B shows mortality assays with minicells derived from P. luminescens strain Kleinii.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### Pesticidal Compositions and Formulations Thereof

An aspect of the disclosure includes a pesticidal composition including a liquid carrier phase; and a plurality of pesticidal minicells dispersed in the carrier phase, wherein the plurality of pesticidal minicells are derived from a plurality of a pesticidal parent bacterium including at least one genetic mutation causing a modification in a cell partitioning function of the parent bacterium, and wherein the plurality of pesticidal minicells are present at a particle concentration sufficient to control at least one pest in or on a plant when the composition is applied to the plant. In some embodiments of this aspect, control includes at least one of: a reduction in pest number on the plant when compared to a check plant not treated with the composition, and a reduction in physical damage to the plant when compared to a check plant not treated with the composition. In some embodiments of this aspect, physical damage includes feeding damage and boring damage. Physical damage may manifest in a variety of plant phenotypes, including but not limited to, chewed or ragged leaves, missing leaves, tunnels in leaves, holes in stems, leaf distortion, leaf discoloration, leaf spotting, wilting, stunted growth, girdled or dead stems, yellowing, breakage damage, or root damage. In further embodiments of this aspect, the reduction in pest number is an at least 10% reduction, an at least 15% reduction, an at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction, and the reduction in physical damage is an at least 10% reduction, an at least 15% reduction, at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction. In other embodiments of this aspect, which may be combined with any of the preceding embodiments, at least a portion of the plurality of pesticidal minicells further include at least one of: an exogenous pesticidal protein toxin, an exogenous pesticidal nucleic acid, and an exogenous pesticidal active ingredient. In some embodiments of this aspect, the portion of the plurality of pesticidal minicells further include an exogenous expression cassette coded to express either or both of the exogenous pesticidal protein toxin and the exogenous pesticidal nucleic acid. The exogenous pesticidal protein toxin, the exogenous pesticidal nucleic acid, or the exogenous pesticidal active ingredient are within the minicell or attached to the minicell membrane. The term “exogenous”, as used herein, includes native proteins expressed by exogenous plasmids. In some embodiments of this aspect, the exogenous pesticidal protein toxin includes at least one of a Pir toxin or a Cry toxin. In some embodiments of this aspect, the exogenous pesticidal nucleic acid is a double-stranded RNA (dsRNA) or a hairpin RNA (hpRNA). In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity. An ingredient with selective herbicidal activity may target parasitic plants, such as broomrape (Orobanche spp.).

In some embodiments of this aspect, which may be combined with any of the preceding embodiments, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC polypeptide, a minD polypeptide, or a minE polypeptide; wherein the cell division topological specificity factor is selected from the group of a minE polypeptide or a DivIVA polypeptide, and wherein the septum machinery component is selected from the group of a ftsZ poly peptide or a ftsA poly peptide. Modification may include overexpression or underexpression (e.g., mutation, deletion, etc.). In some embodiments, the pesticidal parent bacterium includes overexpression of a ftsZ polypeptide. In some embodiments, the pesticidal parent bacterium includes underexpression of a minC polypeptide, a minD poly peptide, and a minE poly peptide.

In some embodiments of this aspect, which may be combined with any of the preceding embodiments, the pesticidal parent bacterium is selected from the group of Streptomyces avermitilis, Saccharopolyspora spinose, Bacillus thuringiensis, Brevibacillus laterosporus, Clostridium bifermentans, Bacillus popilliae, Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, or Escherichia coli. In further embodiments of this aspect, the pesticidal parent bacterium is selected from the group of Bacillus subtilis strain RTI477, Bacillus subtilis strain ATCC 6633, Bacillus subtilis strain ACC 21332, Bacillus subtilis strain 168, Bacillus subtilis strain ATCC 9943, Bacillus subtilis strain QST713, and Bacillus subtilis strain NCB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens IMG 5-29032, Bacillus amyloliquefaciens MBI600, Bacillus amyloliquefaciens CECT8836, or Bacillus amyloliquefaciens M4 (S499). In some embodiments of this aspect, the pesticidal parent bacterium is Photorhabdus luminescens, and wherein the pesticidal minicell includes the exogenous pesticidal protein toxin Pir. In some embodiments of this aspect, the pesticidal parent bacterium is Bacillus subtilis, and wherein the pesticidal minicell includes the exogenous pesticidal molecule. In some embodiments of this aspect, the pesticidal parent bacterium is a genetically modified Escherichia coli expressing one or more exogenous pesticidal active ingredients.

In some embodiments of this aspect, which may be combined with any of the preceding embodiments, the composition is applied to the plant as at least one of a foliar treatment, an injection treatment, a pre-emergence treatment, and a post-emergence treatment. In other embodiments of this aspect, which may be combined with any of the preceding embodiments, the composition is formulated as at least one of a Ready To Use (RTU) formulation, a suspension concentrate (e.g., a liquid flowable formulation), a tank-mix, an aerosol, a seed treatment, a root dip, a soil treatment, an irrigation formulation, a sprinkler formulation, and a drench treatment. In further embodiments of this aspect, the composition is formulated as a dry flowable formulation (e.g., water dispersible granules), a soluble powder formulation, a microencapsulated formulation, or an emulsifiable concentrate formulation. In some embodiments of this aspect, the composition is formulated as the seed treatment. In further embodiments of this aspect, the composition is applied at a rate of about 1×10² to about 1×10⁹ particle/seed, and wherein the rate is determined based on seed size. In further embodiments of this aspect, the composition is applied at a rate of about 1×10⁴ particle/seed. In other embodiments of this aspect, the composition is formulated as the root dip. In further embodiments of this aspect, the composition is applied at a rate of about 1×10³ to about 1×10⁸ particle/plant root system. Further embodiments of this aspect, which may be combined with any of the preceding embodiments, further include agrochemical surfactants, wherein the agrochemical surfactants improve at least one of the characteristics of sprayability, spreadability, and injectability. In further embodiments of this aspect, the liquid carrier phase is aqueous or oil.

Other embodiments of this aspect, which may be combined with any of the preceding embodiments, further include at least one of: an exogenous pesticidal protein toxin, an exogenous pesticidal nucleic acid, and an exogenous pesticidal active ingredient dispersed in the carrier phase. The exogenous pesticidal protein toxin, the exogenous pesticidal nucleic acid, or the exogenous pesticidal active ingredient are in the carrier phase, and are not within the minicell or attached to the minicell membrane. In some embodiments of this aspect, the exogenous pesticidal protein toxin includes a Pir toxin or a Cry toxin. In some embodiments of this aspect, the exogenous pesticidal nucleic acid is a double-stranded RNA (dsRNA) or precursor thereof, a hairpin RNA (hpRNA) or precursor thereof, or a microRNA (miRNA) or precursor thereof. Recombinant miRNA precursors that can be expressed in transgenic plants, and the design of miRNA precursors (e.g., to produce a mature miRNA for cleaving a specific sequence) is disclosed in U.S. Pat. Nos. 7,786,350 and 8,410,334. In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity. An ingredient with selective herbicidal activity may target parasitic plants, such as broomrape (Orobanche spp.). In further embodiments of this aspect, the composition is formulated as the seed treatment. In some embodiments of this aspect, the exogenous pesticidal protein toxin, the exogenous pesticidal nucleic acid, or the exogenous pesticidal active ingredient dispersed in the carrier phase is present in an amount from about 1 g to about 10 g per 100 kg of seed. In some embodiments of this aspect, the exogenous pesticidal protein toxin, the exogenous pesticidal nucleic acid, or the exogenous pesticidal active ingredient dispersed in the carrier phase is present in an amount of about 1×10⁴ particle/seed. In further embodiments of this aspect, the composition is formulated as the root dip. In some embodiments of this aspect, the exogenous pesticidal protein toxin, the exogenous pesticidal nucleic acid, or the exogenous pesticidal active ingredient dispersed in the carrier phase is present from about 25 mg to about 200 mg active ingredient/L. In some embodiments of this aspect, the exogenous pesticidal protein toxin, the exogenous pesticidal nucleic acid, or the exogenous pesticidal active ingredient dispersed in the carrier phase is present in an amount of about 1×10³ to about 1×10³ particle/plant root system. In some embodiments of this aspect, which may be combined with any of the preceding embodiments, the minicell particle concentration is in the range of 1×10² to about 8×10¹⁴. In some embodiments of this aspect, the pesticidal activity of the minicell and the pesticidal activity of the exogenous pesticidal protein toxin, the exogenous pesticidal nucleic acid, or the exogenous pesticidal active ingredient (e.g., in the minicell or attached to the minicell membrane) target the same pest. In other embodiments of this aspect, the pesticidal activity of the minicell and the pesticidal activity of the exogenous pesticidal protein toxin, the exogenous pesticidal nucleic acid, or the exogenous pesticidal active ingredient (e.g., in the minicell or attached to the minicell membrane) target different pests. In some embodiments of this aspect that may be combined with any of the preceding embodiments having an exogenous component dispersed in the carrier phase, the pesticidal activity of the exogenous pesticidal protein toxin, the exogenous pesticidal nucleic acid, or the exogenous pesticidal active ingredient (e.g., in the minicell or attached to the minicell membrane) and the pesticidal activity of the exogenous pesticidal protein toxin, the exogenous pesticidal nucleic acid, or the exogenous pesticidal active ingredient dispersed in the carrier phase target the same pest. In other embodiments of this aspect that may be combined with any of the preceding embodiments having an exogenous component dispersed in the carrier phase, the pesticidal activity of the exogenous pesticidal protein toxin, the exogenous pesticidal nucleic acid, or the exogenous pesticidal active ingredient (e.g., in the minicell or attached to the minicell membrane) and the pesticidal activity of the exogenous pesticidal protein toxin, the exogenous pesticidal nucleic acid, or the exogenous pesticidal active ingredient dispersed in the carrier phase target different pests.

In some embodiments of this aspect, which may be combined with any of the preceding embodiments, the at least one pest is selected from the group of Diamondback moth (DBM). Red flour beetle (RFB). Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichium spp., Botrytis spp., and Cercospora spp. In some embodiments of this aspect, which may be combined with any of the preceding embodiments, the pesticidal minicell remains stable and retains pesticidal activity for at least 8 months, at least 9 months, at least 10 months, at least 11 months, at least 12 months, or at least 13 months.

Still another aspect of the disclosure includes a wettable powder including: a plurality of dried pesticidal minicells derived from a plurality of a pesticidal parent bacterium including at least one genetic mutation causing a modification in a cell partitioning function of the parent bacterium, wherein the wettable powder is configured to be dispersed in an aqueous carrier to create a pesticidal composition for controlling at least one pest in or on a plant when the composition is applied to the plant. Some embodiments of this aspect further include an agrochemically acceptable solid carrier component including at least one of: a clay component, a kaolin component, a talc component, a chalk component, a calcite component, a quartz component, a pumice component, a diatomaceous earth component, a vermiculite component, a silicate component, a silicon dioxide component, a silica powder component, an aluminum component, an ammonium sulfate component, an ammonium phosphate component, a calcium carbonate component, an urea component, a sugar component, a starch component, a sawdust component, a ground coconut shell component, a ground corn cob component, and a ground tobacco stalk component. In some embodiments of this aspect, which may be combined with any of the preceding embodiments, the aqueous carrier includes water. In some embodiments of this aspect, which may be combined with any of the preceding embodiments, control includes at least one of: a reduction in pest number on the plant when compared to a check plant not treated with the composition, and a reduction in physical damage to the plant when compared to a check plant not treated with the composition. In some embodiments of this aspect, physical damage includes feeding damage and boring damage. Physical damage may manifest in a variety of plant phenotypes, including but not limited to, chewed or ragged leaves, missing leaves, tunnels in leaves, holes in stems, leaf distortion, leaf discoloration, leaf spotting, wilting, stunted growth, girdled or dead stems, yellowing, breakage damage, or root damage. In a further embodiment of this aspect, the reduction in pest number is an at least 10% reduction, an at least 15% reduction, an at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction, and the reduction in physical damage is an at least 10% reduction, an at least 15% reduction, at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction. In some embodiments of this aspect, which may be combined with any of the preceding embodiments, the at least one pest is selected from the group of Diamondback moth (DBM). Red flour beetle (RFB), Colorado potato beetle (CPB), Mediterranean flour moth. Fall armyworm (FAW), Asian spotted bollworm, Lepidoptera spp., Coleoptera spp., Diptera spp., Phytophthora spp., Armillaria spp., Colletotrichum spp., Botrytis spp., or Cercospora spp. In other embodiments of this aspect, which may be combined with any of the preceding embodiments, modifying the cell partitioning function of the parent bacterium includes modifying the level or activity of at least one of a z-ring inhibition protein, a cell division topological specificity factor, or a septum machinery component. In further embodiments of this aspect, the z-ring inhibition protein is selected from the group of a minC poly peptide, a minD polypeptide, or a minE polypeptide; wherein the cell division topological specificity factor is selected from the group of a minE polypeptide or a DivIVA polypeptide, and wherein the septum machinery component is selected from the group of a ftsZ polypeptide or a ftsA polypeptide. Modification may include overexpression or underexpression (e.g., mutation, deletion, etc.). In some embodiments, the pesticidal parent bacterium includes overexpression of a ftsZ polypeptide. In some embodiments, the pesticidal parent bacterium includes underexpression of a minC polypeptide, a minD polypeptide, and a minE polypeptide.

A further aspect of the disclosure includes a plantable composition including: a seed; and a coating covering the seed, wherein the coating includes a plurality of pesticidal minicells derived from a plurality of a pesticidal parent bacterium including at least one mutation causing a modification in a cell partitioning function of the parent bacterium, and wherein the plurality of pesticidal minicells are present at a particle concentration sufficient to result in pesticidal activity on at least one pest feeding on the seed or a seedling emerging therefrom. In some embodiments of this aspect, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC polypeptide, a minD poly peptide, or a minE poly peptide; wherein the cell division topological specificity factor is selected from the group of a minE polypeptide or a DivIVA polypeptide, and wherein the septum machinery component is selected from the group of a ftsZ polypeptide or a ftsA polypeptide. Modification may include overexpression or underexpression (e.g., mutation, deletion, etc.). In some embodiments, the pesticidal parent bacterium includes overexpression of a ftsZ poly peptide. In some embodiments, the pesticidal parent bacterium includes underexpression of a minC polypeptide, a minD polypeptide, and a minE polypeptide. In some embodiments of this aspect, which may be combined with any of the preceding embodiments, the pesticidal parent bacterium is selected from the group of Streptomyces avermitilis, Saccharopolvspora spinose, Bacillus thuringiensis. Brevibacillus laterosporus, Clostridium bifermentans, Bacillus popilliae. Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, or Escherichia coli. In further embodiments of this aspect, the pesticidal parent bacterium is selected from the group of Bacillus subtilis strain R77477, Bacillus subtilis strain ATCC 6633, Bacillus subtilis strain ATCC 21332, Bacillus subtilis strain 168, Bacillus subtilis strain ATCC 9943, Bacillus subtilis strain QST713, and Bacillus subtilis strain NCIB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens LMG 5-29032, Bacillus amyloliquefaciens MBI600, Bacillus amyloliquefaciens CECT8836, or Bacillus amyloliquefaciens M4 (S499). In some embodiments of this aspect, which may be combined with any of the preceding embodiments, the coating includes a particle concentration of about 1×10² to about 1×10⁹ particle/seed, and wherein the concentration is determined based on seed size. In further embodiments of this aspect, the particle concentration includes about 1×10⁴ particle/seed. In some embodiments of this aspect, which may be combined with any of the preceding embodiments, the at least one pest is selected from the group of Diamondback moth (DBM), Red flour beetle (RFB). Colorado potato beetle (CPB), Mediterranean flour moth, Fall army worm (FAW), Asian spotted bollworm, Lepidoptera spp., Coleoptera spp., Diptera spp., Phytophthora spp., Armillaria spp., Colletotrichum spp., Botrytis spp., or Cercospora spp. In some embodiments of this aspect, which may be combined with any of the preceding embodiments, the seed is from a plant selected from the group of soybean, strawberry, blackcurrant, white currant, redcurrant, blackberry, raspberry, tomato, pepper, chili, potato, eggplant, cucumber, lettuce, chicory, brassicas, corn, wheat, rice, canola, melon, kale, carrot, or bean.

An additional aspect of the disclosure includes a pesticidal composition including: a pesticidal minicell, wherein the pesticidal minicell is derived from a pesticidal parent bacterium including at least one genetic mutation causing a modification in a level or activity of one or more cell partitioning function factors selected from the group of a minC polypeptide, a minD polypeptide, a minE polypeptide, a ftsZ polypeptide, a ftsA polypeptide, a parA polypeptide, a parB polypeptide, a DivIVA polypeptide, or a combination thereof. Modification may include overexpression or underexpression (e.g., mutation, deletion, etc.). In some embodiments, the pesticidal parent bacterium includes overexpression of a ftsZ polypeptide. In some embodiments, the pesticidal parent bacterium includes underexpression of a minC polypeptide, a minD poly peptide, and a minE poly peptide. In some embodiments of this aspect, the pesticidal minicell includes at least one of: an exogenous pesticidal protein toxin, an exogenous pesticidal nucleic acid, and an exogenous pesticidal active ingredient. In some embodiments of this aspect, the pesticidal minicell further includes an exogenous expression cassette coded to express either or both of the exogenous pesticidal protein toxin and the exogenous pesticidal nucleic acid. The exogenous pesticidal protein toxin, the exogenous pesticidal nucleic acid, or the exogenous pesticidal active ingredient are within the minicell or attached to the minicell membrane. In some embodiments of this aspect, the exogenous pesticidal protein toxin includes at least one of a Pir toxin or a Cry toxin. In some embodiments of this aspect, the exogenous pesticidal nucleic acid is a double-stranded RNA (dsRNA) or a hairpin RNA (hpRNA). In some embodiments of this aspect, the exogenous pesticidal active ingredient is selected from the group of an ingredient with fungicidal activity, an ingredient with insecticidal activity, an ingredient with nematocidal activity, an ingredient with selective herbicidal activity, an ingredient with bactericidal activity, or an ingredient with broad spectrum activity. An ingredient with selective herbicidal activity may target parasitic plants, such as broomrape (Orobanche spp.). In some embodiments of this aspect, which may be combined with any of the preceding embodiments, the pesticidal activity of the minicell and the pesticidal activity of the exogenous pesticidal protein toxin, the exogenous pesticidal nucleic acid, or the exogenous pesticidal active ingredient target the same pest. In other embodiments of this aspect, which may be combined with any of the preceding embodiments, the pesticidal activity of the minicell and the pesticidal activity of the exogenous pesticidal protein toxin, the exogenous pesticidal nucleic acid, or the exogenous pesticidal active ingredient target different pests. In some embodiments of this aspect, which may be combined with any of the preceding embodiments, the at least one pest is selected from the group of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., or Cercospora spp. In some embodiments of this aspect, which may be combined with any of the preceding embodiments, the pesticidal minicell remains stable and retains pesticidal activity for at least 8 months, at least 9 months, at least 10 months, at least 11 months, at least 12 months, or at least 13 months.

The effective amount can be measured by the number of particles, preferably the number of active particles of the pesticidal parent cells or the pesticidal minicell. The number of active particles for a parent cell can be measured by assessing the colony forming units (cfu). The number of active particles for a minicell can be measures by counting the number of minicell vesicles using techniques like flow cytometry.

When used as a seed treatment, the compositions of the present disclosure are applied at a rate of about 1×10² to about 1×10⁹ particles/seed, depending on the size of the seed. In some embodiments, the application rate is 1×10⁴ to about 1×10⁷ particles/seed. In some embodiments, the application rate is about 1×10² to about 1×10⁸, about 1×10² to about 1×10⁷, about 1×10² to about 1×10⁶, about 1×10² to about 1×10⁵, about 1×10² to about 1×10⁴, about 1×10² to about 1×10³, about 1×10³ to about 1×10⁵, or preferably about 1×10⁴ particles/seed. When said compositions are combined or used with at least one additional active ingredient (“ai”), the at least one additional active ingredient may be present in an amount from about 0.001 to about 1000 grams, from about 0.01 to about 500 grams, from about 0.1 to about 300 grams, from about 1 to about 100 grams, from about 1 to about 50 grams, from about 1 to about 25 grams, and preferably from about 1 to about 10 grams per 100 kg of seed, and/or about 1×10² to about 1×10⁸, about 1×10² to about 1×10⁷, about 1×10² to about 1×10⁶, about 1×10² to about 1×10⁵, about 1×10² to about 1×10⁴, about 1×10² to about 1×10³, about 1×10³ to about 1×10⁵, or preferably about 1×10⁴ particles/seed.

The present compositions may also be applied as a root dip at a rate of about 1×10³ to about 1×10⁸ particle/plant root system. When said compositions are combined or used with at least one additional active ingredient, the at least one additional active may be present in an amount from about 0.001 to about 1000 mg, about 0.01 to about 500, about 0.1 to about 400, about 1 to about 300, about 10 to about 250, and preferably from about 25 to about 200 mg ai/L, and/or about 1×10³ to about 1×10⁸ particle/plant root system.

When used as a soil treatment, the compositions of the present disclosure can be applied as a soil surface drench, shanked-in, injected and/or applied in-furrow or by mixture with irrigation water. The rate of application for drench soil treatments, which may be applied at planting, during or after seeding, or after transplanting and at any stage of plant growth, is about 4×10⁷ to about 8×10¹⁴, about 4×10⁹ to about 8×10¹³, about 4×10¹¹ to about 8×10¹² about 2×10¹² to about 6×10¹³, about 2×10¹² to about 3×10¹³, or about 4×10¹³ to about 2×10¹⁴ particle per acre (1.6×10⁷-3.2×10⁴, 1.6×10⁹-3.2×10¹³, 1.6×10¹¹-3.2×10¹², 8×10¹¹-2.4×10¹³, 8×10¹¹-1.2×10¹³ or 1.6×10¹³-8×10¹³ particle per ha). In some embodiments, the rate of application is about 1×10¹² to about 6×10¹² or about 1×10¹³ to about 6×10¹³ particle per acre (4×10¹¹-2.4×10¹² or 4×10¹²-2.4×10¹³ particle per ha). The rate of application for in-furrow treatments, applied at planting, is about 2.5×10¹⁰ to about 5×10¹¹ particle per 1000 row feet (8.3×10⁹-1.7×10¹¹ particle per 100 row m). In some embodiments, the rate of application is about 6×10¹⁰ to about 3×10¹², about 6×10¹⁰ to about 4×10¹¹, about 6×10¹¹ to about 3×10¹², or about 6×10¹¹ to about 4×10¹² particle per 1000 row feet (2×10¹⁰-10¹², 20×10¹⁰-1.3×10¹¹, 2×10¹¹-10¹² or 2×10¹¹-1.3×10¹² particle per 100 row m). The rate of application when shanked or injected into soil is about 4×10⁷ to about 8×10¹⁴, about 4×10¹³ to about 2×10¹⁴ about 4×10⁸ to about 8×10¹³, about 4×10⁹ to about 8×10¹² about 2×10¹⁰ to about 6×10¹¹, about 4×10⁷ to about 8×10¹³, about 4×10⁷ to about 8×10¹², about 4×10⁷ to about 8×10¹¹, about 4×10⁷ to about 8×10¹⁰, about 4×10⁷ to about 8×10⁹, or about 4×10⁷ to about 8×10⁸ particle per acre (1.6×10⁷-3.2×10¹⁴, 1.6×10¹³-8×10¹³, 1.6×10³-3.2×10¹², 1.6×10⁹-3.2×10¹², 8×10⁹-2.4×10¹¹, 1.6×10⁷-3.2×10¹², 1.6×10⁷-3.2×10¹², 1.6×10⁷-3.2×10¹¹, 1.6×10⁷-3.2×10¹⁰, 1.6×10⁷-3.2×10⁹, 1.6×10⁷-3.2×10⁸ particle per ha).

Those of skill in the art will understand how to adjust rates for broadcast treatments (where applications are at a lower rate but made more often) and other less common soil treatments. When said compositions are combined or used with at least one additional active ingredient, the at least one additional active may be present in an amount from about 10 to about 1.000, about 10 to about 750, about 10 to about 500, about 25 to about 500, about 25 to about 250, and preferably from about 50 to about 200 g of ai/ha, and/or about 4×10⁷ to about 8×10¹⁴, about 4×10¹³ to about 2×10¹⁴, about 4×10⁸ to about 8×10¹³, about 4×10⁹ to about 8×10¹² about 2×10¹⁰ to about 6×10¹¹, about 4×10⁷ to about 8×10¹³, about 4×10⁷ to about 8×10¹², about 4×10⁷ to about 8×10¹¹, about 4×10⁷ to about 8×10¹⁰, about 4×10⁷ to about 8×10⁹, or about 4×10⁷ to about 8×10⁸ particle per acre (1.6×10⁷-3.2×10¹⁴, 1.6×10¹³-8×10¹³, 1.6×10⁸-3.2×10¹³, 1.6×10⁹-3.2×10¹², 8×10⁹-2.4×10¹¹, 1.6×10⁷-3.2×10¹³, 1.6×10⁷-3.2×10¹²1.6×10⁷-3.2×10¹¹, 1.6×10⁷-3.2×10¹⁰, 1.6×10⁷-3.2×10⁹, 1.6×10⁷-3.2×10⁸ particle per ha).

The compositions of the present disclosure can be introduced to the soil before planting or before germination of the seed. The compositions of the present disclosure can also be introduced to the loci of the plants, to the soil in contact with plant roots, to soil at the base of the plant, or to the soil around the base of the plant (e.g., within a distance of about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, about 100 cm, or more around or below the base of the plant). The compositions may be applied by utilizing a variety of techniques including, but not limited to, drip irrigation, sprinklers, soil injection or soil drenching. The compositions may also be applied to soil and/or plants in plug trays or to seedlings prior to transplanting to a different plant locus. When applied to the soil in contact with the plant roots, to the base of the plant, or to the soil within a specific distance around the base of the plant, including as a soil drench treatment, the composition may be applied as a single application or as multiple applications. The compositions (including those with at least one additional active ingredient) may be applied at the rates set forth above for drench treatments or at a rate of about 1×10⁵ to about 1×10⁸ particle per gram of soil, 1×10⁵ to about 1×10⁷ particle per gram of soil, 1×10⁵ to about 1×10⁶ particle per gram of soil, 7×10⁵ to about 1×10⁷ particle per gram of soil, 1×10⁶ to about 5×10⁶ particle per gram of soil, or 1×10⁵ to about 3×10⁶ particle per gram of soil, and/or about 4×10⁷ to about 8×10¹⁴, about 4×10⁸ to about 8×10¹³, about 4×10⁹ to about 8×10¹² about 2×10¹⁰ to about 6×10¹¹, about 4×10⁷ to about 8×10¹³, about 4×10⁹ to about 8×10¹², about 4×10¹⁰ to about 8×10¹¹, about 4×10⁷ to about 8×10¹⁰, about 4×10⁷ to about 8×10⁹, or about 4×10⁷ to about 8×10⁸ particle per acre (1.6×10⁷-3.2×10⁴, 1.6×10¹³-8×10¹³, 1.6×10⁸-3.2×10¹³, 1.6×10⁹-3.2×10¹², 8×10⁹-2.4×10¹¹, 1.6×10⁷-3.2×10¹³, 1.6×10⁷-3.2×10¹², 1.6×10⁷ 3.2×10¹¹ 1.6×10⁷-3.2×10¹⁰, 1.6×10⁷-3.2×10¹⁰, 1.6×10-3.2×10⁸ particle per ha). In one embodiment, the compositions of the present disclosure are applied as a single application at a rate of about 7×10⁵ to about 1×10⁷ particle per gram of soil. In another embodiment, the compositions of the present disclosure are applied as a single application at a rate of about 1×10⁶ to about 5×10⁶ particle per gram of soil. In other embodiments, the compositions of the present disclosure are applied as multiple applications at a rate of about 1×10⁵ to about 3×10⁶ particle per gram of soil.

### Methods of Making Pesticidal Minicells

A further aspect of the disclosure includes methods of making pesticidal minicells, including the steps of: (a) providing a pesticidal parent bacterium including at least one genetic mutation causing a modification in a cell partitioning function of the parent bacterium; (b) growing the pesticidal parent bacterium under conditions allowing the formation of pesticidal minicells; and (c) purifying pesticidal minicells using centrifugation, tangential flow filtration (TFF), or TFF and centrifugation. In some embodiments of this aspect, step (c) produces about 10¹⁰ pesticidal minicells per liter, about 10¹¹ pesticidal minicells per liter, about 10¹² pesticidal minicells per liter, about 10¹³ pesticidal minicells per liter, about 10¹⁴ pesticidal minicells per liter, about 10¹⁵ pesticidal minicells per liter, about 10¹⁶ pesticidal minicells per liter, or about 10¹⁷ pesticidal minicells per liter. Some embodiments of this aspect, which may be combined with any of the preceding embodiments, further include step (d) drying the pesticidal minicells to produce a shelf-stable pesticidal minicell composition. In some embodiments of this aspect, the shelf-stable pesticidal minicell composition retains pesticidal activity for at least 8 months, at least 9 months, at least 10 months, at least 11 months, at least 12 months, or at least 13 months. In some embodiments of this aspect, which may be combined with any of the preceding embodiments, the modification in the cell partitioning function of the parent bacterium includes a modification in at least one of a z-ring inhibition protein, a cell division topological specificity factor, or a septum machinery component. In further embodiments of this aspect, the z-ring inhibition protein is selected from the group of a minC polypeptide, a minD polypeptide, or a minE polypeptide; wherein the cell division topological specificity factor is selected from the group of a minE polypeptide or a DivIVA polypeptide, and wherein the septum machinery component is selected from the group of a ftsZ polypeptide or a ftsA polypeptide. Modification may include overexpression or underexpression (e.g., mutation, deletion, etc.). In some embodiments, the pesticidal parent bacterium includes overexpression of a ftsZ polypeptide. In some embodiments, the pesticidal parent bacterium includes underexpression of a minC polypeptide, a minD polypeptide, and a minE polypeptide.

### Pesticidal Minicell-Producing Parent Bacteria

An additional aspect of the disclosure includes a pesticidal minicell-producing parent bacterium, wherein (i) the pesticidal parent bacterium includes a genetic mutation that modifies a cell partitioning function of the parent bacterium; and (ii) the pesticidal parent bacterium exhibits a commercially relevant pesticidal activity with an LD50 against at least one plant pest of less than 100 mg/kg. In other embodiments of this aspect, modifying the cell partitioning function of the parent bacterium includes modifying the level or activity of at least one of a z-ring inhibition protein, a cell division topological specificity factor, or a septum machinery component. In some embodiments of this aspect, which may be combined with any of the preceding embodiments, the z-ring inhibition protein is selected from the group of a minC polypeptide, a minD polypeptide, or a minE polypeptide; wherein the cell division topological specificity factor is selected from the group of a minE polypeptide or a DivIVA polypeptide, and wherein the septum machinery component is selected from the group of a ftsZ polypeptide or a fisA polypeptide. Modification may include overexpression or underexpression (e.g., mutation, deletion, etc.). In some embodiments, the pesticidal parent bacterium includes overexpression of a ftsZ polypeptide. In some embodiments, the pesticidal parent bacterium includes underexpression of a minC polypeptide, a minD polypeptide, and a minE polypeptide. Exemplary pesticidal minicell producing parent bacteria are provided in Tables 1A-1B.

### Methods of Controlling a Pest

Yet another aspect of the disclosure includes methods of controlling a pest, the method including: applying the pesticidal composition of any one of the preceding embodiments to a plant or an area to be planted. In some embodiments of this aspect, the applying includes at least one of an injection application, a foliar application, a pre-emergence application, or a post-emergence application. In some embodiments of this aspect, which may be combined with any of the preceding embodiments, control includes at least one of: a reduction in pest number on the plant when compared to a check plant not treated with the composition, and a reduction in physical damage to the plant when compared to a check plant not treated with the composition. In some embodiments of this aspect, physical damage includes feeding damage and boring damage. Physical damage may manifest in a variety of plant phenotypes, including but not limited to, chewed or ragged leaves, missing leaves, tunnels in leaves, holes in stems, leaf distortion, leaf discoloration, leaf spotting, wilting, stunted growth, girdled or dead stems, yellowing, breakage damage, or root damage. In further embodiments of this aspect, the reduction in pest number is an at least 10% reduction, an at least 15% reduction, an at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction, and the reduction in physical damage is an at least 10% reduction, an at least 15% reduction, at least 20% reduction, an at least 25% reduction, an at least 30% reduction, an at least 35% reduction, an at least 40% reduction, an at least 45% reduction, an at least 50% reduction, an at least 55% reduction, an at least 60% reduction, an at least 65% reduction, an at least 70% reduction, an at least 75% reduction, or an at least 80% reduction. In other embodiments of this aspect, which may be combined with any of the preceding embodiments, the composition is formulated as at least one of a Ready To Use (RTU) formulation, a suspension concentrate, a tank-mix, an aerosol, a seed treatment, a root dip, a soil treatment, an irrigation formulation, a sprinkler formulation, or a drench treatment. In some embodiments of this aspect, which may be combined with any of the preceding embodiments, the pest is selected from the group of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., or Cercospora spp.

### Definitions

The term “control,” as used herein, means killing, reducing in numbers, and/or reducing growth, feeding or normal physiological development of any or all life stages of a plant pest, and/or reduction of the effects of a plant pest infection and/or infestation. An effective amount is an amount able to noticeably reduce pest growth, feeding, root penetration, maturation in the root, and/or general normal physiological development and/or symptoms resulting from the plant pest infection. In some embodiments the symptoms resulting from the plant pest infection and/or the number of plant pest particles are reduced by at least about 5%, at least about 10%, at least about 20%, at least about 30%, at least about 40%, at least about 50%, at least about 60%, at least about 70%, at least about 80%, or at least about 90% versus untreated controls.

For nematode pests, the term “control.” as used herein, means killing, reducing in numbers, and/or reducing growth, feeding or normal physiological development of any or all life stages of nematodes (including, for root knot nematodes, the ability to penetrate roots and to develop within roots), reduction of the effects of nematode infection and/or infestation (e.g., galling, penetration, and/or development within roots), resistance of a plant to infection and/or infestation by nematodes, resistance of a plant to the effects of nematode infection and/or infestation (e.g., galling and/or penetration), tolerance of a plant to infection and/or infestation by nematodes, tolerance of a plant to the effects of nematode infection and/or infestation (e.g., galling and/or penetration), or any combination thereof. The resistance to and tolerance of plants to parasitic nematodes has been known to those of ordinary skill in the art, as demonstrated by Trudgill, D. L. “Resistance to and Tolerance of Plant Parasitic Nematodes in Plants.” Annual Review of Phytopathology. 1991; 29:167-192, which is specifically and entirely incorporated by reference herein for everything it teaches. An effective amount is an amount able to noticeably reduce pest growth, feeding, root penetration, maturation in the root, and/or general normal physiological development and symptoms resulting from nematode infection. In some embodiments symptoms and/or nematodes are reduced by at least about 5%, at least about 10%, at least about 20%, at least about 30%, at least about 40%, at least about 50%, at least about 60%, at least about 70%, at least about 80%, or at least about 90% versus untreated controls.

The term “minicell” refers to a achromosomal, non-replicating, enclosed membrane system including at least one membrane and having an interior volume suitable for containing a cargo (e.g., one or more of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). Minicells are achromosomal cells that are products of aberrant cell division, and contain RNA and protein, but little or no chromosomal DNA. Minicells are capable of plasmid-directed synthesis. Minicells can be derived from a parent bacterial cell (e.g., a gram-negative or a gram-positive bacterial cell) using preferably genetic manipulation of the parent cell which—for example—disrupt the cell division machinery of the parent cell. In some embodiments, the minicell may include one or more endogenous or heterologous features of the parent cell surface, e.g., cell walls, cell wall modifications, flagella, or pili, and/or one or more endogenous or heterologous features of the interior volume of the parent cell, e.g., nucleic acids, plasmids, proteins, small molecules, transcription machinery, or translation machinery. In other embodiments, the minicell may lack one or more features of the parent cell. In still other embodiments, the minicell may be loaded or otherwise modified with a feature not included in the parent cell.

“Pesticidal minicell” refers to a minicell obtained from a pesticidal parent bacterial cell. In a preferred embodiment the pesticidal minicells retains all or part of the pesticidal activity of the patent bacterial cell.

“Pesticidal parent bacterial cell” refers to a parent bacterial cell with a direct toxic activity on a plant pest. Direct toxic activity means the ability to cause death to a plant pest without the necessity of an interaction with the crop plant. In a preferred embodiment the LD50 of the pesticidal parent cell is less than 100 mg/kg. LD50 is the amount of a material, given all at once, which causes the death of 50% (one half) of a group of test target pest organisms.

As used herein, the term “parent bacterial cell” refers to a cell (e.g., a gram-negative or a gram-positive bacterial cell) from which a minicell is derived. Parent bacterial cells are typically viable bacterial cells. The term “viable bacterial cell” refers to a bacterial cell that contains a genome and is capable of cell division. Preferred parent bacterial cells are provided in Table 2A. The parent bacterial cell includes at least one genetic mutation causing a modification in a cell partitioning function of the parent bacterium.

The term “cell division topological specificity factor” refers to a component of the cell division machinery in a bacterial species that is involved in the determination of the site of the septum and functions by restricting the location of other components of the cell division machinery, e.g., restricting the location of one or more Z-ring inhibition proteins. Exemplary cell division topological specificity factors include minE, which was first discovered in E. coli and has since been identified in a broad range of gram negative bacterial species and gram-positive bacterial species (Rothfield et al., Nature Reviews Microbiology, 3: 959-968, 2005), minE functions by restricting the Z-ring inhibition proteins minC and minD to the poles of the cell. A second exemplary cell division topological specificity factor is DivIVA, which was first discovered in Bacillus subtilis (Rothfield et al., Nature Reviews Microbiology, 3: 959-968, 2005).

The term “Z-ring inhibition protein” refers to a component of the cell division machinery in a bacterial species that is involved in the determination of the site of the septum and functions by inhibiting the formation of a stable FtsZ ring or anchoring such a component to a membrane. The localization of Z-ring inhibition proteins may be modulated by cell division topological specificity factors, e.g., MinE and DivIVA. Exemplary Z-ring inhibition proteins include minC and minD, which were first discovered in E. coli and have since been identified in a broad range of gram-negative bacterial species and gram-positive bacterial species (Rothfield et al., Nature Reviews Microbiology, 3: 959-968, 2005). In E. coli and in other species, minC, minD, and minE occur at the same genetic locus, which may be referred to as the “min operon”, the minCDE operon, or the min or minCDE genetic locus.

### ENUMERATED EMBODIMENTS

The following enumerated embodiments are representative of some aspects of the invention.

1. A pesticidal composition comprising:
a liquid carrier phase; and
a plurality of pesticidal minicells dispersed in the carrier phase,
wherein the plurality of pesticidal minicells are derived from a plurality of a pesticidal parent bacterium comprising at least one genetic mutation causing a modification in a cell partitioning function of the parent bacterium, and
wherein the plurality of pesticidal minicells are present at a particle concentration sufficient to control at least one pest in or on a plant when the composition is applied to the plant.

2. The pesticidal composition of embodiment 1, wherein control includes at least one of:
a reduction in pest number on the plant when compared to a check plant not treated with the composition, and
a reduction in physical damage to the plant when compared to a check plant not treated with the composition.

3. The pesticidal composition of embodiment 2, wherein
the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, at least 20% reduction, an at least 25% reduction, an at least 30% reduction, an at least 40% reduction, an at least 50% reduction, an at least 60% reduction, an at least 70% reduction, or an at least 80% reduction.

4. The pesticidal composition of any one of embodiments 1-3, wherein at least a portion of the plurality of pesticidal minicells further comprise at least one of: an exogenous pesticidal protein toxin, an exogenous pesticidal nucleic acid, and an exogenous pesticidal active ingredient.

5. The pesticidal composition of embodiment 4, wherein the portion of the plurality of pesticidal minicells further comprise an exogenous expression cassette coded to express either or both of the exogenous pesticidal protein toxin and the exogenous pesticidal nucleic acid.

6. The pesticidal composition of embodiment 4 or 5, wherein the exogenous pesticidal protein toxin comprises at least one of a Pir toxin and a Cry toxin.

7. The pesticidal composition of embodiment 4 or 5, wherein the exogenous pesticidal nucleic acid is a double-stranded RNA (dsRNA) or a hairpin RNA (hpRNA).

8. The pesticidal composition of embodiment 4, wherein the exogenous pesticidal active ingredient is selected from the group consisting of an ingredient with fungicidal activity, an ingredient with insecticidal activity, an ingredient with nematocidal activity, an ingredient with selective herbicidal activity, an ingredient with bactericidal activity, and an ingredient with broad spectrum activity.

9. The pesticidal composition of any one of embodiments 1-8, wherein the modification in the cell partitioning function of the parent bacterium includes a modification in at least one of a z-ring inhibition protein, a cell division topological specificity factor, or a septum machinery component.

10. The pesticidal composition of embodiment 9, wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA polypeptide, and wherein the septum machinery component is selected from the group consisting of a ftsZ polypeptide and a ftsA polypeptide.

11. The pesticidal composition of any one of embodiments 1-10, wherein the pesticidal parent bacterium is selected from the group consisting of Streptomyces avermitilis, Saccharopolyspora spinose, Bacillus thuringiensis, Brevibacillus laterosporus, Clostridium bifermentans, Bacillus popilliae, Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, and Escherichia coli.

12. The pesticidal composition of embodiment 11, wherein the pesticidal parent bacterium is selected from the group consisting of Bacillus subtilis strain RTI477, Bacillus subtilis strain ATCC 6633, Bacillus subtilis strain ATCC 21332, Bacillus subtilis strain 168, Bacillus subtilis strain ATCC 9943, Bacillus subtilis strain QST713, and Bacillus subtilis strain NCIB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens LMG 5-29032, Bacillus amyloliquefaciens MBI600, Bacillus amyloliquefaciens CECT8836, and Bacillus amyloliquefaciens M4 (S499).

13. The pesticidal composition of any one of embodiments 1-11, wherein the pesticidal parent bacterium is Photorhabdus luminescens, and wherein the pesticidal minicell comprises the exogenous pesticidal protein toxin Pir.

14. The pesticidal composition of any one of embodiments 1-11, wherein the pesticidal parent bacterium is Bacillus subtilis, and wherein the pesticidal minicell comprises the exogenous pesticidal molecule.

15. The pesticidal composition of any one of embodiments 1-10, wherein the pesticidal parent bacterium is a genetically modified Escherichia coli expressing one or more exogenous pesticidal active ingredients.

16. The pesticidal composition of any one of embodiments 1-15, wherein the composition is applied to the plant as at least one of a foliar treatment, an injection treatment, a pre-emergence treatment, and a post-emergence treatment.

17. The pesticidal composition of any one of embodiments 1-16, wherein the composition is formulated as at least one of a Ready To Use (RTU) formulation, a suspension concentrate, a tank-mix, an aerosol, a seed treatment, a root dip, a soil treatment, an irrigation formulation, a sprinkler formulation, and a drench treatment.

18. The pesticidal composition of embodiment 17, wherein the composition is formulated as the seed treatment.

19. The pesticidal composition of embodiment 18, wherein the composition is applied at a rate of about 1×10² to about 1×10⁹ particle/seed, and wherein the rate is determined based on seed size.

20. The pesticidal composition of embodiment 19, wherein the composition is applied at a rate of about 1×10⁴ particle/seed.

21. The pesticidal composition of embodiment 17, wherein the composition is formulated as the root dip.

22. The pesticidal composition of embodiment 21, wherein the composition is applied at a rate of about 1×10³ to about 1×10⁸ particle/plant root system.

23. The pesticidal composition of any one of embodiments 1-22, further comprising agrochemical surfactants, wherein the agrochemical surfactants improve at least one of the characteristics of sprayability, spreadability, and injectability.

24. The pesticidal composition of any one of embodiments 1-23, wherein the liquid carrier phase is aqueous or oil.

25. The pesticidal composition of any one of embodiments 1-24, further comprising at least one of: an exogenous pesticidal protein toxin, an exogenous pesticidal nucleic acid, and an exogenous pesticidal active ingredient dispersed in the carrier phase.

26. The pesticidal composition of embodiment 25, wherein the exogenous pesticidal protein toxin comprises a Pir toxin and a Cry toxin.

27. The pesticidal composition of embodiment 25, wherein the exogenous pesticidal nucleic acid is a double-stranded RNA (dsRNA) or precursor thereof, a hairpin RNA (hpRNA) or precursor thereof, or a microRNA (miRNA) or precursor thereof.

28. The pesticidal composition of embodiment 25, wherein the exogenous pesticidal active ingredient is selected from the group consisting of an ingredient with fungicidal activity, an ingredient with insecticidal activity, an ingredient with nematocidal activity, an ingredient with selective herbicidal activity, an ingredient with bactericidal activity, and an ingredient with broad spectrum activity.

29. The pesticidal composition of any one of embodiments 25-28, wherein the composition is formulated as the seed treatment.

30. The pesticidal composition of embodiment 29, wherein the exogenous pesticidal protein toxin, the exogenous pesticidal nucleic acid, or the exogenous pesticidal active ingredient dispersed in the carrier phase is present in an amount from about 1 g to about 10 g per 100 kg of seed.

31. The pesticidal composition of embodiment 29, wherein the exogenous pesticidal protein toxin, the exogenous pesticidal nucleic acid, or the exogenous pesticidal active ingredient dispersed in the carrier phase is present in an amount of about 1×10⁴ particle/seed.

32. The pesticidal composition of any one of embodiments 25-28, wherein the composition is formulated as the root dip.

33. The pesticidal composition of embodiment 32, wherein the exogenous pesticidal protein toxin, the exogenous pesticidal nucleic acid, or the exogenous pesticidal active ingredient dispersed in the carrier phase is present from about 25 mg to about 200 mg active ingredient/L.

34. The pesticidal composition of embodiment 32, wherein the exogenous pesticidal protein toxin, the exogenous pesticidal nucleic acid, or the exogenous pesticidal active ingredient dispersed in the carrier phase is present in an amount of about 1×10³ to about 1×10³ particle/plant root system.

35. The pesticidal composition of any one of embodiments 1-34, wherein the minicell particle concentration is in the range of about 1×10² to about 8×10¹⁴.

36. The pesticidal composition of any one of embodiments 4-35, wherein the pesticidal activity of the minicell and the pesticidal activity of the exogenous pesticidal protein toxin, the exogenous pesticidal nucleic acid, or the exogenous pesticidal active ingredient target the same pest.

37. The pesticidal composition of any one of embodiments 4-35, wherein the pesticidal activity of the minicell and the pesticidal activity of the exogenous pesticidal protein toxin, the exogenous pesticidal nucleic acid, or the exogenous pesticidal active ingredient target different pests.

38. The pesticidal composition of any one of embodiments 25-37, wherein the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target the same pest.

39. The pesticidal composition of any one of embodiments 25-37, wherein the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target different pests.

40. The pesticidal composition of any one of embodiments 1-39, wherein the at least one pest is selected from the group consisting of Diamondback moth (DBM), Red flour beetle (RFB), Colorado potato beetle (CPB), Mediterranean flour moth. Fall armyworm (FAW). Asian spotted bollworm, Lepidoptera spp., Coleoptera spp., Diptera spp., Phytophthora spp., Armillaria spp., Colletotrichum spp., Botrytis spp., and Cercospora spp.

41. The pesticidal composition of any one of embodiments 1-40, wherein the pesticidal minicell remains stable and retains pesticidal activity for at least 8 months, at least 9 months, at least 10 months, at least 11 months, at least 12 months, or at least 13 months.

42. A method of making pesticidal minicells, comprising the steps of:
a) providing a pesticidal parent bacterium comprising at least one genetic mutation causing a modification in a cell partitioning function of the parent bacterium;
b) growing the pesticidal parent bacterium under conditions allowing the formation of pesticidal minicells; and
c) purifying pesticidal minicells using centrifugation, tangential flow filtration (TFF), or TFF and centrifugation.

43. The method of embodiment 42, wherein step (c) produces about 10¹⁰ pesticidal minicells per liter, about 10¹¹ pesticidal minicells per liter, about 10¹² pesticidal minicells per liter, about 10¹³ pesticidal minicells per liter, about 10¹⁴ pesticidal minicells per liter, about 10¹⁵ pesticidal minicells per liter, about 10¹⁶ pesticidal minicells per liter, or about 10¹⁷ pesticidal minicells per liter.

44. The method of embodiment 42 or embodiment 43, further comprising step (d) drying the pesticidal minicells to produce a shelf-stable pesticidal minicell composition.

45. The method of embodiment 44, wherein the shelf-stable pesticidal minicell composition retains pesticidal activity for at least 8 months, at least 9 months, at least 10 months, at least 11 months, at least 12 months, or at least 13 months.

46. The method of any one of embodiments 4245, wherein the modification in the cell partitioning function of the parent bacterium includes a modification in at least one of a z-ring inhibition protein, a cell division topological specificity factor, or a septum machinery component.

47. The method of embodiment 46, wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA polypeptide, and wherein the septum machinery component is selected from the group consisting of a ftsZ polypeptide and a ftsA polypeptide.

48. A pesticidal minicell-producing parent bacterium, wherein
(i) the pesticidal parent bacterium comprises a genetic mutation that modifies a cell partitioning function of the parent bacterium; and
(ii) the pesticidal parent bacterium exhibits a commercially relevant pesticidal activity with an LD50 against at least one plant pest of less than 100 mg/kg.

49. The pesticidal minicell-producing parent bacterium of embodiment 48, wherein modifying the cell partitioning function of the parent bacterium includes modifying the level or activity of at least one of a z-ring inhibition protein, a cell division topological specificity factor, or a septum machinery component.

50. The pesticidal minicell-producing parent bacterium of embodiment 48 or embodiment 49, wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA polypeptide, and wherein the septum machinery component is selected from the group consisting of a ftsZ polypeptide and a fisA poly peptide.

51. A method of controlling a pest, the method comprising:
applying the pesticidal composition of any one of embodiments 1-41 to a plant or an area to be planted.

52. The method of embodiment 51, wherein the applying includes at least one of an injection application, a foliar application, a pre-emergence application, or a post-emergence application.

53. The method of embodiment 51 or embodiment 52, wherein control includes at least one of:
a reduction in pest number on the plant when compared to a check plant not treated with the composition, and
a reduction in physical damage to the plant when compared to a check plant not treated with the composition.

54. The method of embodiment 53, wherein the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction.

55. The method of any one of embodiments 51-54, wherein the composition is formulated as at least one of a Ready To Use (RTU) formulation, a suspension concentrate, a tank-mix, an aerosol, a seed treatment, a root dip, a soil treatment, an irrigation formulation, a sprinkler formulation, or a drench treatment.

56. The method of any one of embodiments 51-55, wherein the pest is selected from the group consisting of Diamondback moth (DBM), Red flour beetle (RFB), Colorado potato beetle (CPB), Mediterranean flour moth. Fall armyworm (FAW), Asian spotted bollworm, Lepidoptera spp., Coleoptera spp., Diptera spp., Phytophthora spp., Armillaria spp., Colletotrichum spp., Botrytis spp., and Cercospora spp.

57. A wettable powder comprising:
a plurality of dried pesticidal minicells derived from a plurality of a pesticidal parent bacterium comprising at least one genetic mutation causing a modification in a cell partitioning function of the parent bacterium,
wherein the wettable powder is configured to be dispersed in an aqueous carrier to create a pesticidal composition for controlling at least one pest in or on a plant when the composition is applied to the plant.

58. The wettable powder of embodiment 57, further comprising an agrochemically acceptable solid carrier component comprising at least one of: a clay component, a kaolin component, a talc component, a chalk component, a calcite component, a quartz component, a pumice component, a diatomaceous earth component, a vermiculite component, a silicate component, a silicon dioxide component, a silica powder component, an aluminum component, an ammonium sulfate component, an ammonium phosphate component, a calcium carbonate component, an urea component, a sugar component, a starch component, a sawdust component, a ground coconut shell component, a ground corn cob component, and a ground tobacco stalk component.

59. The wettable powder of embodiment 57 or embodiment 58, wherein the aqueous carrier comprises water.

60. The wettable powder of any one of embodiments 57-59, wherein control includes at least one of:
a reduction in pest number on the plant when compared to a check plant not treated with the composition, and
a reduction in physical damage to the plant when compared to a check plant not treated with the composition.

61. The wettable powder of embodiment 60, wherein
the reduction in pest number is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction, and the reduction in physical damage is an at least 10% reduction, an at least 15% reduction, an at least 20% reduction, an at least 25% reduction, an at least 30% reduction, an at least 40% reduction, an at least 50% reduction, an at least 60% reduction, an at least 70% reduction, or an at least 80% reduction.

62. The wettable powder of any one of embodiments 57-61, wherein the at least one pest is selected from the group consisting of Diamondback moth (DBM). Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., and Cercospora spp.

63. The wettable powder of any one of embodiments 57-62, wherein modifying the cell partitioning function of the parent bacterium includes modifying the level or activity of at least one of a z-ring inhibition protein, a cell division topological specificity factor, or a septum machinery component.

64. The wettable powder of embodiment 63, wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA poly peptide, and wherein the septum machinery component is selected from the group consisting of a ftsZ polypeptide and a ftsA polypeptide.

65. A plantable composition comprising:
a seed; and
a coating covering the seed, wherein the coating comprises a plurality of pesticidal minicells derived from a plurality of a pesticidal parent bacterium comprising at least one mutation causing a modification in a cell partitioning function of the parent bacterium, and
wherein the plurality of pesticidal minicells are present at a particle concentration sufficient to result in pesticidal activity on at least one pest feeding on the seed or a seedling emerging therefrom.

66. The plantable composition of embodiment 65, wherein the modification in the cell partitioning function of the parent bacterium includes a modification in at least one of a z-ring inhibition protein, a cell division topological specificity factor, or a septum machinery component.

67. The plantable composition of embodiment 66, wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA polypeptide, and wherein the septum machinery component is selected from the group consisting of a ftsZ polypeptide and a fisA polypeptide.

68. The plantable composition of any one of embodiments 65-67, wherein the pesticidal parent bacterium is selected from the group consisting of Streptomyces avermitilis, Saccharopolyspora spinose, Bacillus thuringiensis, Brevibacillus laterosporus, Clostridium bifermentans, Bacillus popilliae, Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, and Escherichia coli.

69. The plantable composition of embodiment 68, wherein the pesticidal parent bacterium is selected from the group consisting of Bacillus subtilis strain RTI477, Bacillus subtilis strain ATCC 6633, Bacillus subtilis strain ATCC 21332, Bacillus subtilis strain 168, Bacillus subtilis strain ATCC 9943, Bacillus subtilis strain QST713, and Bacillus subtilis strain NCIB 3610, Bacillus atrophaeus strain ABI02A DSM 32019, Bacillus atrophaeus strain ABI03 DW 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens LMG 5-29032, Bacillus amyloliquefaciens MBI600, Bacillus amyloliquefaciens CECT8836, and Bacillus amyloliquefaciens M4 (S499).

70. The plantable composition of any one of embodiments 65-69, wherein the coating comprises a particle concentration of about 1×10² to about 1×10⁹ particle/seed, and wherein the concentration is determined based on seed size.

71. The plantable composition of embodiment 70, wherein the particle concentration comprises about 1×10⁴ particle/seed.

72. The plantable composition of any one of embodiments 65-71, wherein the at least one pest is selected from the group consisting of Diamondback moth (DBM), Red flour beetle (RFB), Colorado potato beetle (CPB). Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., and Cercospora spp.

73. The plantable composition of any one of embodiments 65-72, wherein the seed is from a plant selected from the group consisting of soybean, strawberry, blackcurrant, white currant, redcurrant, blackberry, raspberry, tomato, pepper, chili, potato, eggplant, cucumber, lettuce, chicory, brassicas, corn, wheat, rice, canola, melon, kale, carrot, and bean.

74. A pesticidal composition comprising:
a pesticidal minicell, wherein the pesticidal minicell is derived from a pesticidal parent bacterium comprising at least one genetic mutation causing a modification in a level or activity of one or more cell partitioning function factors selected from the group consisting of a minC polypeptide, a minD polypeptide, a minE polypeptide, a ftsZ polypeptide, a ftsA polypeptide, a parA polypeptide, a parB polypeptide, a DivIVA polypeptide, and a combination thereof.

75. The pesticidal composition of 74, wherein the pesticidal minicell comprises at least one of: an exogenous pesticidal protein toxin, an exogenous pesticidal nucleic acid, and an exogenous pesticidal active ingredient.

76. The pesticidal composition of embodiment 75, wherein the pesticidal minicell further comprises an exogenous expression cassette coded to express either or both of the exogenous pesticidal protein toxin and the exogenous pesticidal nucleic acid.

77. The pesticidal composition of embodiment 75 or embodiment 76, wherein the exogenous pesticidal protein toxin comprises a Pir toxin or a Cry toxin.

78. The pesticidal composition of embodiment 75 or embodiment 76, wherein the exogenous pesticidal nucleic acid is a double-stranded RNA (dsRNA) or a hairpin RNA (hpRNA).

79. The pesticidal composition of embodiment 75, wherein the exogenous pesticidal active ingredient is selected from the group consisting of an ingredient with fungicidal activity, an ingredient with insecticidal activity, an ingredient with nematocidal activity, an ingredient with selective herbicidal activity, an ingredient with bactericidal activity, and an ingredient with broad spectrum activity.

80. The pesticidal composition of any one of embodiments 74-79, wherein the pesticidal activity of the minicell and the pesticidal activity of the exogenous pesticidal protein toxin, the exogenous pesticidal nucleic acid, or the exogenous pesticidal active ingredient target the same pest.

81. The pesticidal composition of any one of embodiments 74-79, wherein the pesticidal activity of the minicell and the pesticidal activity of the exogenous pesticidal protein toxin, the exogenous pesticidal nucleic acid, or the exogenous pesticidal active ingredient target different pests.

82. The pesticidal composition of any one of embodiments 74-81, wherein the at least one pest is selected from the group consisting of Diamondback moth (DBM), Red flour beetle (RFB), Colorado potato beetle (CPB), Mediterranean flour moth, Fall armyworm (FAW), Asian spotted bollworm, Lepidoptera spp., Coleoptera spp., Diptera spp., Phytophthora spp., Armillaria spp., Colletotrichum spp., Botrytis spp., and Cercospora spp.

83. The pesticidal composition of any one of embodiments 74-82, wherein the pesticidal minicell remains stable and retains pesticidal activity for at least 8 months, at least 9 months, at least 10 months, at least 11 months, at least 12 months, or at least 13 months.

### EXAMPLES

The presently disclosed subject matter will be better understood by reference to the following Examples, which are provided as exemplary of the invention, and not by way of limitation.

### Example 1: Production of a Pesticidal Minicell by Genetic Modifications

This example shows that pesticidal minicells may be produced from pesticidal parent bacterial cells by various genetic mutations. In this example, methods of producing pesticidal minicells are provided that include disruption of one or more genes involved in regulating the cell partitioning function of the parent bacterium, i.e., disruption of a z-ring inhibition protein (e.g., minC or minD) or disruption of z-ring inhibition proteins and a cell division topological specificity factor (e.g., minCDE). Additionally, the genetic means of creating ADAS-producing strains via disruption of the min operon or over-expression of the septum machinery component FtsZ is provided.

### Materials and Methods

### Bioinformatics Identification of Target Genes

Photorhabdus luminescens strains TT01 and Kleinii: The sequences for genes of interest were found on the database PhotoList World-wide Web Server (http://genolist.pasteur.fr/PhotoList/genome.cgi) supported by the Institut Pasteur.

Bacillus subtilis subsp. inaquosorum: To identify the sequences to disrupt in a species without a genome sequence, the following procedure is taken. rDNA is amplified from the chromosome by PCR using primers Primers017 and Primers046 and sequenced via Sanger sequencing (Table 1). The closest sequenced relative strain is then identified using nucleotide BLAST. The genome sequence of this closest relative is used to identify the genes involved in minicell formation (divIVA, minC, minD), and to design primers targeting the disruption of these loci. Additionally, the sequence of the master regulator of sporulation, spo0A, is identified and used to design primers to amplify homology regions for genetic disruption.

TABLE 1
Primer list and sequences

| Primer name | Sequence |
|---|---|
| oAF086 | TGGTAATCTATGTATCCTGGCAAC (SEQ ID NO: 1) |
| oLK015 | TTCGCCAGATGATAAGGAAC (SEQ ID NO: 2) |
| oLK092 | GGTCTCGgcattctcgcaatattatccatcctgcc (SEQ ID NO: 3) |
| oLK111 | taGGTCTCgtctcgatataaaggcacaaagcgg (SEQ ID NO: 4) |
| oZC017 | ATACTTGTCCACTTTGCACCG (SEQ ID NO: 5) |
| oZC046 | TTCGGGTAGACAAATTGCAC (SEQ ID NO: 6) |

Production of Pesticidal Minicells from Photorhabdus luminescens Strains Via ftsZ Over-Expression and Min Mutations

Production of minicells via overexpression of ftsZ protein: Pesticidal minicells were produced from Photorhabdus luminescens strains TT01 and Kleinii by the overexpression of the native ftsZ protein. A BsaI gBlock was ordered containing the sequence and the native ribosomal binding site (RBS). Using Golden Gate assembly, the gBlock was moved into expression vector Pla071 (CloDF origin containing ampicillin resistance and the TetR promoter induced by aTc). The resulting expression vector was Pla097 and was transformed into E. coli DH5α via heat shock with chemically competent cells. Once that was complete, the plasmid was miniprepped out of the E. coli strain and then transformed into the Photorhabdus luminescens strains. The Photorhabdus luminescens strains were grown in CASO medium, washed in 5%[w/v] Sucrose+1 mM HEPES buffer, plated on CASO+Carbenicillin 50 μg/mL, and grown at 30° C. for two days. Colonies that grew on the recovery plates were picked and colony PCR was conducted to test for proper plasmid propagation. Primers oLK015 (SEQ ID NO: 2) and oAF086 (SEQ ID NO: 1) were used to verify the existence of the P. luminescens gene ftsZ and successful plasmid transformation.

For the production of minicells, a plate was streaked from the frozen glycerol stock and incubated for two days at 30° C. Colonies were picked and inoculated in LB+Carb 50 to grow at 30° C. overnight. The overnight culture was diluted the next morning at 1:200 in a larger volume of media plus antibiotic. The culture grew until OD600 0.5 was reached, then the culture was induced with 100 ng/1 mL of aTc, and grown overnight. The next day, the culture was processed to collect minicells that were produced overnight following a differentiation centrifugation process described in Example 2.

Production of minicells via deletion of minCDE operon: In order to knock out the operon, cells are transfected with a suicide plasmid. The plasmid, pPINT, is acquired from the Ralf Heermann lab at Johannes-Gutenberg-Universität Mainz, Institut für Molekulare Physiologie. The pPINT plasmid has a multiple cloning site (MCS) that allows for the addition of homology arms for the insertion of an antibiotic cassette. The homology arms are created using primers, with cut site overhangs matching the pPINT MCS, that bound to the genome. One arm is set to contain 500 bp upstream of minC, and the other arm is set to contain 500 bp downstream of minE. Using restriction digest and ligation, the final pPINT plasmid is then transformed into an auxotrophic donor pir+ E. coli strain. This is necessary due to the R6K origin of the plasmid. Once the donor strain has the plasmid, the transfection protocol is started. The transfection leads to homologous recombination to insert the antibiotic cassette where the minCDE operon is located. The Photorhabdus strain is plated on Kan35 to select for the proper antibiotic resistance from the transfection. Following this first level of selection, the colonies are placed on sucrose containing agar to select against the donor strain. PCR verification is executed using primers that land outside the homology arm and a primer that lands inside the antibiotic cassette. Minicells are produced because the minCDE operon is removed and there is no regulation of cellular division. Without minCDE the division of the cell is uncontrolled; the ftsZ ring will form at the pole of the bacteria and create the minicell when it cinches closed. For minicell production, a fresh plate from the glycerol freezer stock is streaked and incubate overnight at 30° C. The following day, a single colony is inoculated in a 50 mL LB culture. The culture is grown at 30° C. overnight and the next day, 10 mL of the overnight culture is taken and inoculated in 500 mL culture. This process is repeated to have a total of one (1) liter of material between two flasks. As stated above, the minicells will be produced due to the minCDE mutation. The next day the culture is processed to collect minicells that are produced overnight following a differentiation centrifugation processes described in Example 2.

Production of Pesticidal Minicells from Bacillus subtilis Subsp. Inaquosorum Via Min Mutations

To produce pesticidal minicells of B. subtilis subsp. inaquosorum, genomic deletions of divIVA and minCD are produced. The divIVA and/or minCD locus is replaced with an antibiotic resistance gene encoding for either kanamycin resistance or erythromycin resistance flanked by loxP recombination sites (following a strategy similar to Koo, et al 2017). The master regulator of sporulation, spo0A, is also deleted to prevent formation of spores, which would compete with the formation of minicells and are of similar size, making minicell purification more cumbersome. Briefly, 1 kb regions upstream and downstream of the divIVA, minCD, or spo0A locus are amplified by PCR. These homology arms are then sewn to the gene encoding the antibiotic resistance marker and loxP sites via PCR to create a knockout cassette. The cassette is then transformed into B. subtilis subsp. inaquosorum following standard transformation procedures.

The resistance marker is then removed through the use of the Cre-lox recombinase system. Briefly, strains containing disruptions in the divIVA, minCD, and spo0A loci are transformed with the plasmid pDR422, which encodes for a constitutively expressed Cre recombinase gene and a temperature-sensitive origin of replication, and plated on spectinomycin selective LB-agar plates at 30° C. The following day, several colonies are restreaked onto non-selective LB-agar plates and incubated at 37° C. to remove the pDR422 plasmid. Resulting colonies are patched onto kanamycin, erythromycin, spectinomycin, and plain LB-agar plates to confirm loss of all antibiotic resistances.

Production of minicells is confirmed by observation of minicells in a culture of Bacillus subtilis subsp. inaquosorum. Briefly, a single colony of mutant B. subtilis is picked and grown in LB at 37° C. for 3 hours, or until an OD₆₀₀=1 is reached. 1 μL of culture is then placed onto a pad on a microscope slide made of 1.5% agarose in PBS. A cover slip is placed on top, and the culture is imaged with a 100× oil immersion objective by phase contrast light microscopy.

### Results

Table 2A, below, provides the pesticidal parent bacterial cells, the closest relative bacteria, and the classification. Table 2B provides the proteins identified in the pesticidal parent bacterial cells for production of minicells.

TABLE 2A
Pesticidal parent cells, closest relative bacteria, and classification.

| | Closest | | | | |
|---|---|---|---|---|---|
| Pesticidal | relative | Classification | | | |
| parent cells | bacteria | Family | Order | Class | Phylum |
| Streptomyces | | Streptomyce- | Actinomycetales | Actinobacteria | Actinobacteria |
| avermitilis | | taceae | | | |
| Saccharo- | Saccharo- | Pseudonocar- | Actinomycetales | Actinobacteria | Actinobacteria |
| polyspora | polyspora | diaceae | | | |
| spinosa | erythrea | | | | |
| Bacillus | | Bacillaceae | Bacillales | Bacilli | Firmicutes |
| thuringiensis | | | | | |
| Brevibacillus | | Paenibacillaceae | Bacillales | Bacilli | Firmicutes |
| laterosporus | | | | | |
| Clostridium | Clostridium | Clostridiaceae | Clostridiales | Clostridia | Firmicutes |
| bifermentans | botulinum | | | | |
| Bacillus | Paenibacillus | Bacillaceae | Bacillales | Bacilli | Firmicutes |
| popilliae | polymyxa | | | | |
| Escherichia | | Entero- | Entero- | Gammaproteo- | Proteobacteria |
| coli | | bacteriaceae | bacteriales | bacteria | |
| Photorhabdus | Photorhabdus | Entero- | Entero- | Gammaproteo- | Proteobacteria |
| luminescens | asymbiotica | bacteriaceae | bacteriales | bacteria | |
| Xenorhabdus | | Entero- | Entero- | Gammaproteo- | Proteobacteria |
| nematophila | | bacteriaceae | bacteriales | bacteria | |
| Serratia | Serratia | Entero- | Entero- | Gammaprote- | Proteobacteria |
| entomophila | marcescens | bacteriaceae | bacteriales | obacteria | |
| Yersinia | Yersinia | Yersiniaceae | Entero- | Gammaproteo- | Proteobacteria |
| entomophaga | enterocolitica | | bacteriales | bacteria | |
| Pseudomonas | | Pseudomona- | Pseudomona- | Gammaproteo- | Proteobacteria |
| entomophila | | daceae | dales | bacteria | |
| Burkholderia | Burkholderia | Burkholder- | Burkholderiales | Betaproteo- | Proteobacteria |
| spp. | mallei | iaceae | | bacteria | |
| Chromo- | Chromo- | Neisseriaceae | Neisseriales | Betaproteo- | Proteobacteria |
| bacterium | bacterium | | | bacteria | |
| subtsugae | violaceum | | | | |

TABLE 2B
Proteins identified in the pesticidal parent bacterial cells for
production of minicells (Y = protein is present).

| | Cell division proteins | | | | | | |
|---|---|---|---|---|---|---|---|
| Pesticidal parent cells | minC | minD | minE | ftsZ | ftsA | parA | parB |
| Streptomyces avermitilis | | | | Y | | Y | Y |
| Saccharopolyspora | | | | Y | | Y | Y |
| spinosa | | | | | | | |
| Bacillus thuringiensis | Y | Y | | Y | Y | Y | Y |
| Brevibacillus | Y | Y | | Y | Y | Y | Y |
| laterosporus | | | | | | | |
| Clostridium | Y | Y | Y | Y | Y | Y | Y |
| bifermentans | | | | | | | |
| Bacillus popilliae | Y | Y | | Y | Y | Y | Y |
| Escherichia coli | Y | Y | Y | Y | Y | | |
| Photorhabdus | Y | Y | Y | Y | Y | | |
| luminescens | | | | | | | |
| Xenorhabdus | Y | Y | Y | | Y | Y | Y |
| nematophila | | | | | | | |
| Serratia entomophila | Y | Y | Y | Y | Y | Y | |
| Yersinia entomophaga | Y | Y | Y | Y | Y | Y | Y |
| Pseudomonas | Y | Y | Y | Y | Y | Y | Y |
| entomophila | | | | | | | |
| Burkholderia spp. | Y | Y | Y | Y | Y | Y | Y |
| Chromobacterium | Y | Y | Y | Y | Y | Y | Y |
| subtsugae | | | | | | | |

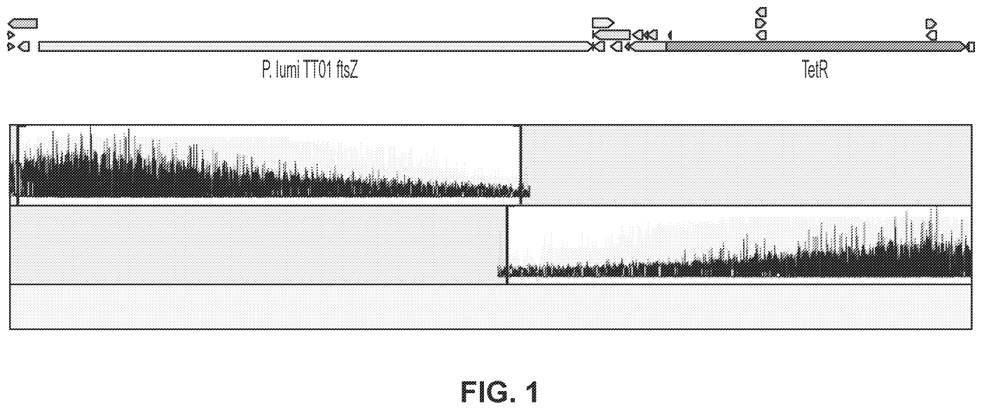

FIG. 1 provides a sequencing map showing that the Photorhabdus luminescens ftsZ gene was successfully inserted into the expression vector.

### Example 2: Isolation and Characterization of Pesticidal Minicells

Several methods may be used to purify minicells from the parental bacterial culture. This example describes three methods for minicell isolation: a centrifugation process, tangential flow filtration (TFF) process, and combined centrifugation-TFF process. In all cases, antibiotic treatment is used to sterilize the minicell culture.

### Materials and Methods

### Media Optimization

LB was used as the base line, and a study was done to identify more optimal media for fermentation. Several medias were tested for minicell production of the bacterial strains. Medias tested included Defined Media with different carbon sources (Cas Amino Acids/Yeast Extract/Peptone), LB, and TB+2% glycerol. The study was done with 50 mL of material in a 250 mL flask following the minicell protocol. The purified minicell samples were measured with the Spectradyne and the results were compared.

### Culturing of Minicell Producing Strains

To produce minicells, a plate was streaked from the frozen glycerol stock and incubated for two days at 30° C. Colonies were picked and inoculated an overnight culture in LB+Carb 50 to grow at 30° C. overnight. The overnight culture was diluted the next morning at 1:200 in a larger volume of media plus antibiotic. The culture was grown until OD600 0.5 was reached, then the culture was induced with 100 ng/mL of aTc, and grown overnight. The next day, the culture was processed to collect minicells that were produced overnight following a differentiation centrifugation processes described below.

Minicell producing strains of B. subtilis are grown in rich media (LB), in 1-liter cultures in 2.5 L shake flasks at 37° C. with shaking at 250 rpm. The culture is inoculated by selection of a single colony from a fresh LB-agar plate and cultured for 12, 16, 18, or 24 hrs.

### Centrifugation Particles Purification

Briefly, bacterial cultures are diluted to an OD600=10, and centrifuged in 1-liter bottles at 4000×g (Sorval Lynx 6000) for 40 minutes using the slowest acceleration speed. The minicell rich supernatant is then centrifuged at 17,000 g for 1 hour to pellet minicells. The resulting pellet is then resuspended in 50 mL of fresh LB containing 200 μg/mL ceftriaxone and 20 μg/mL ciprofloxacin, and the culture is placed at 30° C. for 2 hours to remove any remaining parental bacteria. The solution is centrifuged in a swinging bucket rotor (Beckman Coulter) at 4,000×g for 15 min to remove the dead parental bacterial cells and large debris. The minicells are then pelleted at 20,000×g (Sorval Lynx 6000) for 20 min and resuspended in an equal volume of 0.2 μm-filtered PBS. This step is repeated for a total of 2 washes, and the resulting minicell pellet is resuspended in a final volume of 1 mL of 0.2 μm-filtered PBS.

### TFF Particles Purification

Briefly, the majority of parental cells are removed via a first tangential-flow filtration (TFF) using a 0.65 μM filter and collecting the permeate without concentration. Contaminants are then removed and the minicell rich permeate concentrated 10-fold via use of a 750 kDa TFF filter, collecting the retentate. The minicell rich retentate is then treated with 200 μg/mL ceftriaxone and 20 μg/mL ciprofloxacin, and the culture is placed at 30° C. for 2 hours to remove any remaining parental bacteria and then processed as described above.

### Centrifugation-TFF Particles Purification

This method of minicell isolation combines steps from “Centrifugation particles purification” and “TFF particles purification”. Briefly, the culture is diluted to an OD600=10 and parental cells removed via centrifugation as in “Centrifugation particles purification”. The minicell rich supernatant is then purified of contaminants and concentrated via TFF through use of a 750 kDa filter as in “TFF particles purification”. The minicell rich retentate is then treated with antibiotics, washed, and concentrated as in “Centrifugation particles purification” and “TFF particles purification”.

### Characterization of Minicells

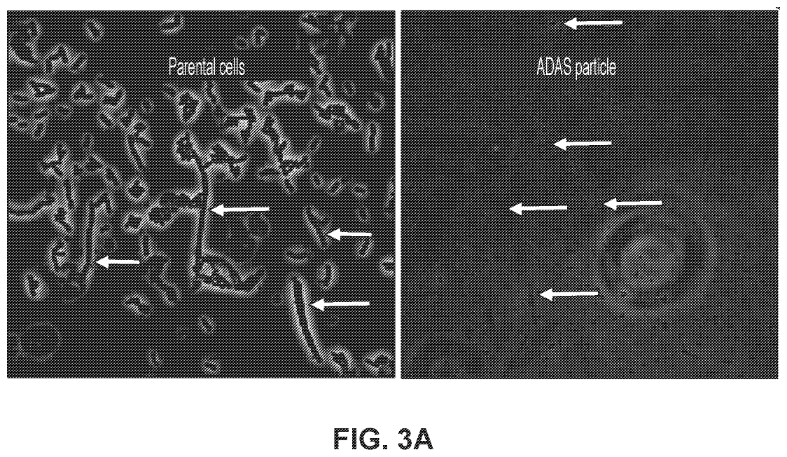

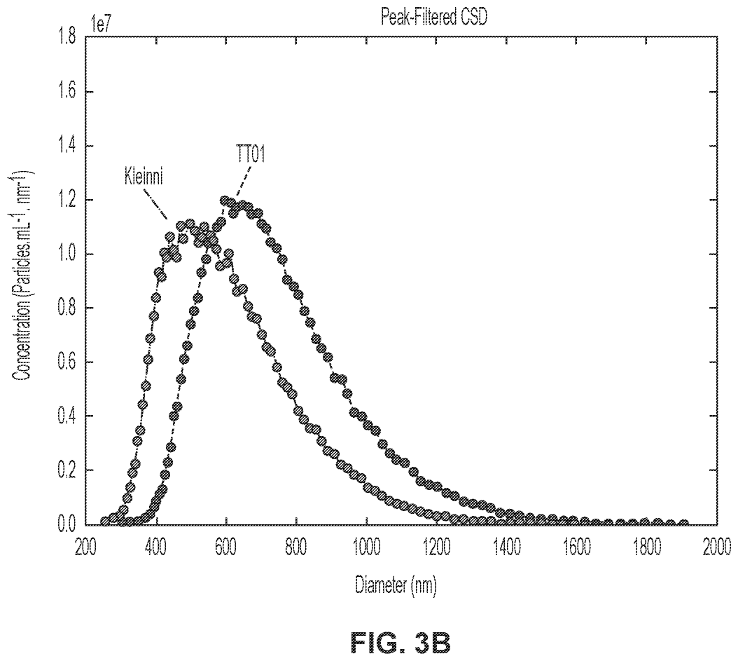

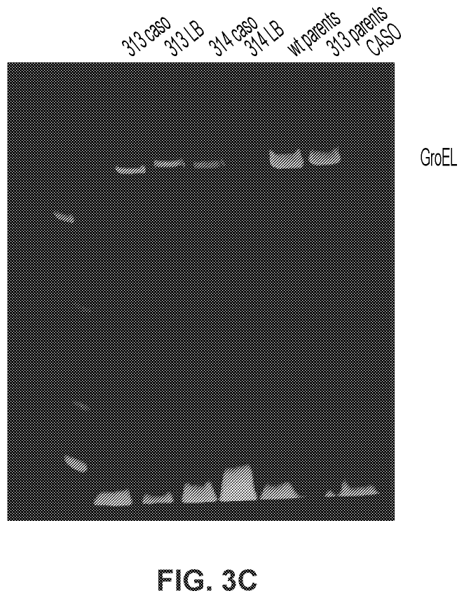

Isolated minicells were validated by microscopy, particle size analysis, and western blotting for the cytosolic protein GroEL. FIG. 3A shows a phase contrast microscopy image of a culture of a minicell producing P. luminescens strain before and after minicell isolation. FIG. 3D shows particle size analysis results. Particle size distribution and concentration were measured by counting with a Spectradyne nCS1.

To confirm the minicells were true minicells and not extracellular vesicles, which there are reports Photorhabdus species produce, western blotting for the cytosolic chaperone GroEL was performed. Briefly, 3.33 μL of 1×LDS sample buffer (Thermo) were mixed with 10 μL of sample containing 1e9 particles. Samples were boiled at 95° C. for 5 min in 1.5 mL tubes and spun in a benchtop centrifuge to remove any condensation from the lid. The entire sample was loaded onto a Bolt 4-12% Bis-Tris PAGE gel (Thermo). 7 μL of Chameleon Duo ladder (LiCOR) were loaded as a standard. Proteins were separated on the gel by running at a constant 200 V for 25 min. The proteins were then transferred to a nitrocellulose membrane using an iBlot system (Thermo) using the V0 program. Following transfer, the membrane was incubated with a mouse anti-GroEL antibody (Abcam ab82592) and goat anti-mouse Alexa Fluor 647 (Thermo A32728) via an iBind (Thermo) using manufacturer recommended antibody dilutions. The blot was then imaged with an iBright imager (Thermo) using smart exposure settings for Alexa Fluor 647. FIG. 3C shows an image of a western blot for cytosolic chaperone GroEL.

### Results

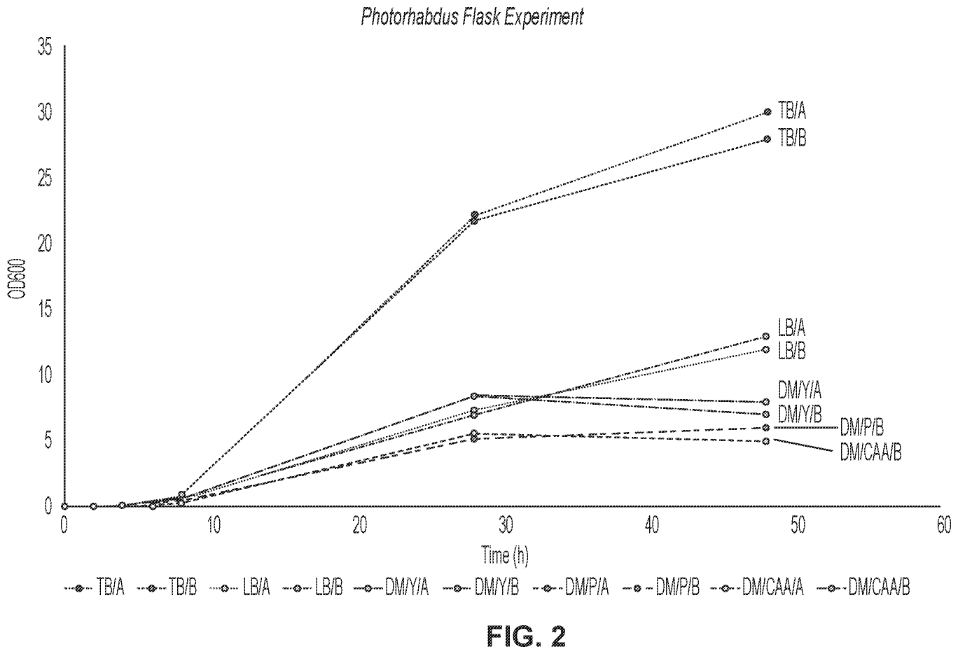

FIG. 2 shows the results of the media optimization testing.

FIGS. 3A-3C show assays characterizing pesticidal minicells produced from Photorhabdus luminescens. FIG. 3A is a microscopy image in which spherical minicell particles of ˜500 nm are clearly visible (on right). FIG. 3D shows particle size analysis results, which showed that concentrations of greater than 1e10, 1e11, and 1e12 per liter were collected from a 1 L culture, with an average size of 450 nm. FIG. 3C shows an image of a western blot for cytosolic chaperone GroEL. It can be seen that only minicells were positive for GroEL, whereas extracellular vesicles were not, as they only contain periplasmic material.

### Example 3: Treatment of Plants with a Pesticidal Composition Including Pesticidal Minicells Derived from Pesticidal Parent Bacteria Kill Insect Pests while Preserving Plant Health

This example demonstrates the ability to kill or decrease the fitness of the insect Plutella xylostella (Diamondback Moth), by treating them with a pesticidal composition including pesticidal minicells derived from the entomopathogenic microbe Photorhabdus luminescens. This example also demonstrates that this treatment results in diminished plant damage in susceptible plants.

### Materials and Methods

### Insect Rearing

P. xylostella eggs were purchased from Benzon Research and are reared on an artificial diet (general noctuid diet) purchased from Benzon Research. The diet was prepared as follows:
1. 162 g of the general noctuid diet powder was added to boiling water
2. The contents were mixed thoroughly for 15 minutes while keeping the temperature between 80° C. and 90° C.
3. The mixture was cooled down to 70° C., 5 mL of linseed oil was added and mixed in thoroughly
4. The food was then dispensed into rearing containers and allowed to cool and solidify

The DBM eggs were placed on the diet and allowed to hatch and feed. All rearing containers were maintained at 25° C., 16 hour:8 hour light:dark cycle, and 34% humidity. Once the larvae reached 2nd instar stage, they were used for artificial diet or leaf disk assays. At this stage, the larvae can also be used for whole plant assays.

### Insect Experimental Treatment Using Artificial Diet, Leaf Disk Assay and Whole Plants

For artificial diet assays, 0.38 mL of noctuid diet was dispensed into each well of a 48-well plate, cooled, and stored at 4° C. overnight. The following day, 30 μL of compositions of 10⁸, 10⁹, 10¹⁰, or 10¹¹ insecticidal minicells (prepared as in Example 2), or sterile PBS as a negative control, were layered on top of the diet in each well. After drying for 1 hour in a fume hood, one 2nd instar stage DBM larvae was placed into each well. The plate was then taped firmly with a Breathe Easier sheet (Qiagen) and placed in an incubator maintained at 25° C. with 16 hour:8 hour light:dark cycle, and 34% humidity.

For leaf disk assays, leaf disks were made from canola leaves with a circular leather cutter. Each leaf disk was then placed on top of 1% autoclaved agar gel in a 12-well plate. Images of each plate were taken with a Lemnatec imager prior to minicell composition application and insect infestation. To facilitate spreading, Silwet L-77 was added to all minicell solutions to a final concentration of 0.05%. 25 μL of solutions containing 10⁸, 10⁴, 10¹⁰, or 10¹¹ of P. luminescens minicells, or PBS as a negative control, were then dispensed onto the leaf disks and allowed to dry completely. After drying, five 2nd instar DBM larvae were placed on each leaf disk. The plates were sealed with a Breathe Easier sheet and placed in an incubator maintained at 25° C. with 16 hour:8 hour light:dark cycle, and 34% humidity.

For whole plant assays, three leaf stage canola plants are used. Fresh DBM eggs are left in a hatching chamber overnight with a wet paper towel. 12 canola plants are placed in each cage before being sprayed with a minicell solution. They are allowed to dry for 1 hour before infesting. The paper towel from the rearing chamber (containing hundreds of neonates) is cut in half and placed in each cage.

Plant Health and Insect Fitness Readouts after Treatments (LD50 Assays)

The effect of pesticidal minicells on insect fitness in artificial diet or leaf disk assays was determined after 3 days. Alive larvae were counted and developmental stages determined. Mortality and developmental stunting were determined. The LD50 of P. luminescens minicells on DBM larvae was determined by plotting the larval survival percentage against the number of minicells applied to artificial diet and fitting a dose-response curve (GraphPad Prism 9).

For leaf disk assays, photos of leaf disk plates were taken with a Lemnatech imager and the percentage of leaf disk consumed was calculated.

For whole plant assays, larval size and damage to plants is documented through daily observation and photos. The number of pupated and non-pupated larvae are counted at the end of the experiment and the weight of each plant is measured to assess the effect of the minicells on DBM survival and feeding.

### Results

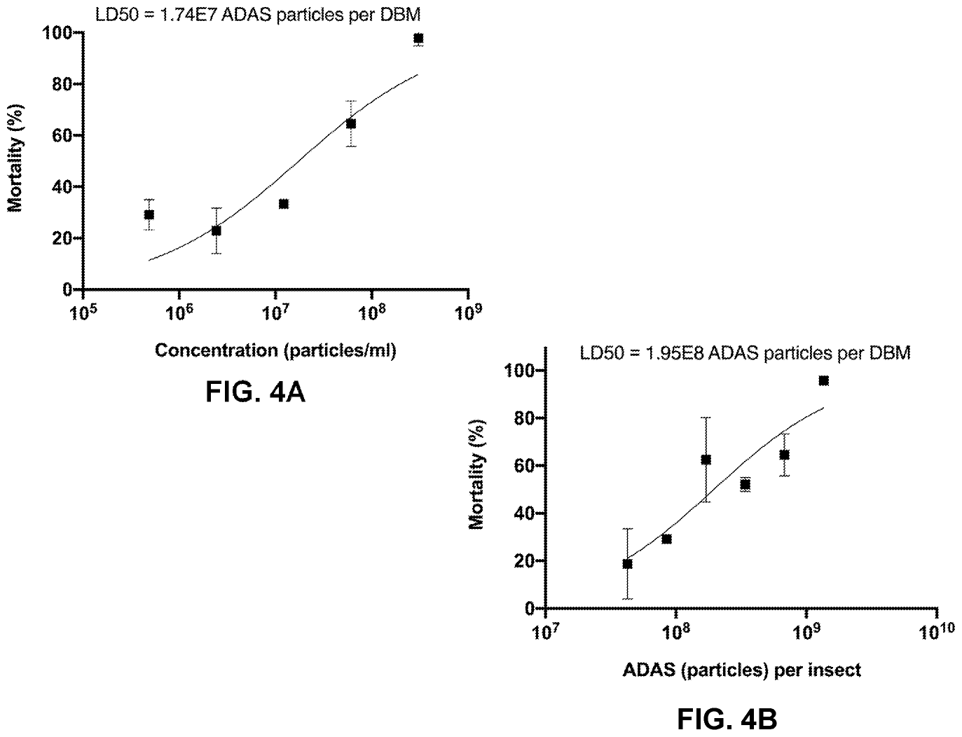

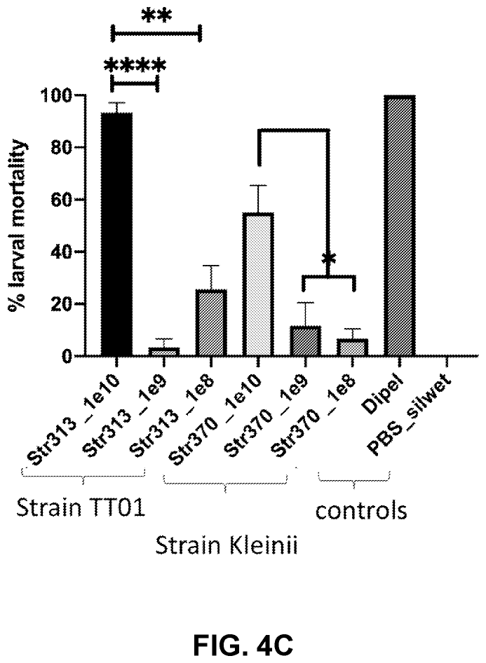

FIGS. 4A-4C show the results of LD50 assays in which Plutella xylostella (Diamondback Moth; DBM) were treated with pesticidal compositions containing minicells produced from P. luminescens. FIGS. 4A-4B show the results of artificial diet LD50 assays in which DBM larvae were fed a series of concentrations of the minicell particles derived from P. luminescens strain TT01 (FIG. 4A) or Kleinni (FIG. 4B). Mortality was recorded 3 days after feeding. FIG. 4C shows the results of a leaf disk assay LD50 assay in which DBM larvae were fed a series of concentrations of the minicell particles derived from P. luminescens strains TT01 or Kleinii and mortality was recorded 3 days later. In these assays, pesticidal minicells demonstrated mortality and strong stunting phenotypes.

### Example 4: Treatment of a Panel of Lepidopteran Insects with a Pesticidal Composition Including Pesticidal Minicells Derived from Pesticidal Parent Bacteria, Show High Susceptibility of P. xylostella

This example demonstrates the ability to specifically kill or decrease the fitness of the insect Plutella xylostella (Diamondback Moth), but not other insect larvae, by treating them with a pesticidal composition including pesticidal minicells derived from the entomopathogenic microbe Photorhabdus luminescens.

### Materials and Methods

### Insect Rearing and Experimental Treatment Using Artificial Diet

European Corn Borer (Ostrinia nubilalis; ECB) and Fall Army Worm (Spodoptera frugiperda; FAW) eggs were obtained from Benzon Research Inc. Insect rearing was conducted as in Example 3. Artificial diet assays were set up as in Example 3.

Insect Fitness Readouts after Treatments (% Mortality)

Readouts of insect fitness and mortality were performed as in Example 3.

### Results

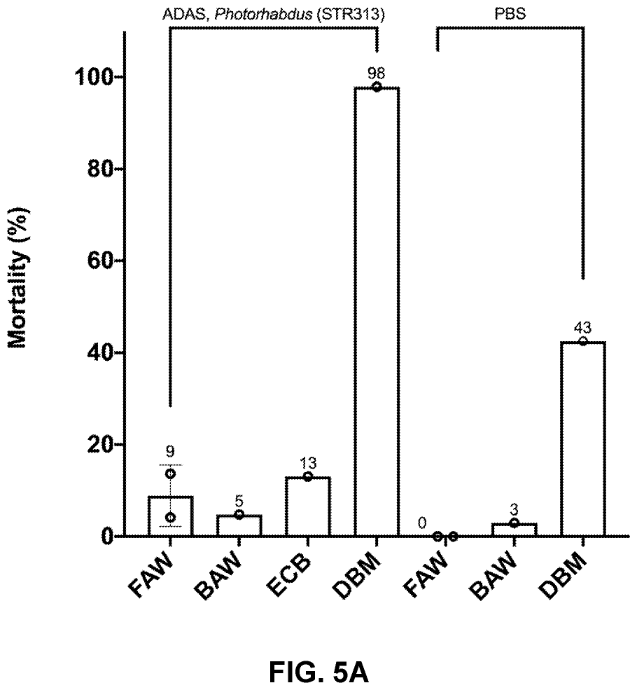

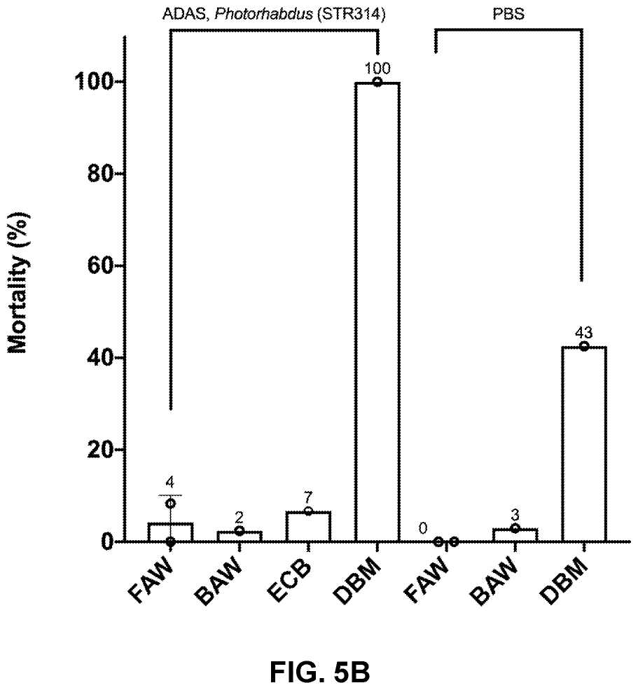

FIGS. 5A-5B show that the pesticidal minicells from P. luminescens were toxic to Diamondback Moth (DBM), but not to Fall Army Worm (FAW). Beet Army Worm (BAW), and European Corn Borer (ECB).

### Example 5: Production of Pesticidal Minicells Further Including an Exogenous Insecticidal Active Ingredient

This example demonstrates encapsulation of Chlorantraniliprole (CTPR) and loading concentration quantification (encapsulation efficacy).

### Materials and Methods

Pesticidal minicells from P. luminescens E1.2 (500 μL) were eluted in PBS. Either the pesticidal minicells or PBS solution (500 μL) was spiked in with 5 μL of CTPR stock (10 mg/ml), then incubated in an incubator at 37 C for 24 hrs. Then 100 μL sample (spiked pesticidal minicells or PBS) was then subjected to centrifugal filtration process with a filter (Microcon-300 kDa, EMD Millipore). The samples were washed 6 times with sterile 1% MeOH in PBS and centrifuged for 6 times at 15,000 g for 1 min to remove free A.I. After the 6th filtration, all the filtrates were collected in one tube as total filtrate. Additional 100 μL 1% MeOH in PBS was added to filter to wash and recover the retentate (ADAS) from the filter. Both retentate (pesticidal minicells) and filtrates were subjected to LC-MS to detect the concentration of CTPR.

### Example 6: Insecticidal Potency and Spectrum Increase of a Pesticidal Minicell Derived from Pesticidal Parent Bacteria by Encapsulation of an Exogenous Insecticidal Active Ingredient

### Materials and Methods

### Insects Rearing (DBM, FAW, ECB)

Insect rearing of DBM, ECB, and FAW larvae are conducted as in Example 4.

### Experimental Treatment

Insects are treated with the pesticidal minicells of Example 5 in artificial diet and LDA assays

### Insect Fitness Readouts

Insect fitness readouts after treatments (LDA assays) are conducted as in Example 4.

### Example 7: Treatment of Plants with a Pesticidal Composition Including Pesticidal Minicells Derived from a Fungicidal Parent Bacterium, Inhibit Fungi′ Pests Preserving Plant Health

This example demonstrates the ability to inhibit the fungi Botrytis cinerea that causes the disease Botrytis gray mold, by treating them with a pesticidal minicells derived from the fungicidal microbe Bacillus subtilis subsp. inaquosorum. This example also demonstrates that this treatment results in a diminished plant and fruit damage in susceptible plants.

### Materials and Methods

### Fungi Culture and Experimental Treatment Using In Vitro Assays

Antifungal activity is demonstrated by a hyphal zone of inhibition assay. A lawn of B. cinerea is grown at room temperature on a potato dextrose agar (PDA) plate for 1 week. A plug of this lawn is placed in the center of a fresh PDA plate, and filter disks coated in at least 10⁸, 10⁹, 10¹⁰, minicells of B. subtilis subsp. inaquosorum are arranged equidistant from the fungal plug. The plate is imaged after 5 and 7 days, and a zone of inhibition is measured in mm.

Plant Health and Fungi Inhibition Readouts after Treatments (LD50 Assays)

The effectiveness of topically applied pesticidal minicells from B. subtilis subsp. inaquosorum in inhibiting grey mold diseases is tested under greenhouse conditions. Briefly, pesticidal minicells are applied with multiple dilutions with a starting concentration of 10\hat{ }10 minicells before strawberries are inoculated with the pathogen B. cinerea. After, multiple samplings are done a different time points (1, 6, 24 hr and 7 days) of the strawberry fruits from plants for testing disease severity. Lesion diameters in the fruit are then compared between treatments.

### Example 8: Production of Pesticidal Minicells Further Including an Exogenous Fungicidal Active Ingredient

### Materials and Methods

### Encapsulation of Azoxystrobin and Loading Concentration Quantification (Encapsulation Efficacy)

Pesticidal minicells from B. subtilis subsp. inaquosorum E1.3 (500 μL) were eluted in PBS. Either the pesticidal minicells or PBS solution (500 μL) was spiked in with 5 μL of azoxystrobin stock (10 mg/ml), then incubated in an incubator at 37 C for 24 hrs. Then 100 μL sample (spiked pesticidal minicells or PBS) was then subjected to centrifugal filtration process with a filter (Microcon-300 kDa, EMD Millipore). The samples were washed 6 times with sterile 1% MeOH in PBS and centrifuged for 6 times at 15,000 g for 1 min to remove free A.I. After the 6th filtration, all the filtrates were collected in one tube as total filtrate. Additional 100 μL 1% MeOH in PBS was added to filter to wash and recover the retentate (pesticidal minicells) from the filter. Both retentate (pesticidal minicells) and filtrates are subjected to LC-MS to detect the concentration of the A.I.

### Example 9: Fungicidal Potency and Spectrum Increase of a Pesticidal Minicells Derived from a Fungicidal Parent Bacterium by Encapsulation of a Fungicidal Chemical Agent

### Materials and Methods

### Fungi Culture (Botrytis, Fusarium)

Fungi growth is conducted as in Example 7.

### Experimental Treatment Using Pesticidal Minicells Further Including an Exogenous Fungicidal Active Ingredient of Example 8 In Vitro and LDA Assays

Treatments and read outs are conducted as in Example 7.

### Example 10: Production of Pesticidal Minicells Derived from Fungicidal Parent Bacteria Further Including an Exogenous Insecticidal Active Ingredient

### Materials and Methods

Encapsulation of CTPR into Minicells from B. subtilis and Loading Concentration Quantification (Encapsulation Efficacy)

Encapsulation and quantification of the incorporated A.I is done as in Example 5.

### Results

### Example 11: Pesticidal Spectrum Increase Using Pesticidal Minicells Derived from a Fungicidal Parent Bacteria Including an Exogenous Insecticidal Active Ingredient

### Materials and Methods

### Fungi Culture (Botrytis)

Fungi growth is conducted as in Example 7.

### Insect Rearing (DBM)

Insect rearing of DBM larvae are conducted as in Example 3.

### Experimental Treatment Using Pesticidal Minicells of Example 10, In Vitro and Artificial Diet Assays

Readouts of insect fitness and mortality are performed as in Example 3.

Fungal Inhibition and Insect Fitness Readouts after Treatments

Treatments and read outs are conducted as in Example 7.

### Example 12: Production of Storage-Stable Pesticidal Minicells

### Materials and Methods

### Pesticidal Minicells Lyophilization Process (Both Photorhabdus and Bacillus Derived)

This example demonstrates the ability to create a storage-stable pesticidal minicells that maintains activity.

To create a storage-stable pesticidal minicells in this example, minicells are freeze-dried via lyophilization. Isolated minicells of Photorhabdus luminescens TT01 or Bacillus subtilis in PBS are prepared as in Example 2, and 1 mL of minicells are pelleted by centrifugation at 21,000 g for 15 min in 1.5 mL plastic tubes. The pellet is resuspended in and equal volume of Microbial Freeze Drying Buffer (OPS Diagnostics) is transferred into 15 mL conical tube, and flash frozen in liquid nitrogen. The pesticidal minicells are then freeze-dried for 16 hours using a FreeZone benchtop freeze dryer (Labconco) with autocollect settings. Tubes of freeze dried minicells are sealed with parafilm and stored at room temperature in the dark until use.

### Storage of Pesticidal Minicells and Assay of Stability

Freeze-dried minicells are stored for a period of 1, 2, 6, 12, or 24 months. Activity is measure after hydration. Briefly, powdered minicells are rehydrated with 1 mL of PBS. Maintenance of particle numbers is confirmed by concentration measurement on a Spectradyne nCS1. ATP content of minicells is measure as well to confirm stability.

### Example 13: Creation of a Wettable Powder (WP) Pesticidal Composition

### Materials and Methods

Creation of a WP Using the Lyophilized Minicells from Example 12

A lyophilized pesticidal minicell as produced before may be used to make a wettable powder (WP) according to the disclosure. Wettable powders as used herein include finely divided particles that disperse readily in water or other liquid carriers. The particles contain pesticidal minicells, typically in lyophilized form, retained in a solid matrix. Typical solid matrices include fuller's earth, kaolin clays, silicas and other readily wet organic or inorganic solids. Wettable powders normally contain about 5% to about 95% of the active ingredient plus a small amount of wetting, dispersing or emulsifying agent.

### Results

Exemplary wettable powders could include those in Table 3, below.

TABLE 3
Exemplary wettable powders.

| Ingredient | Example 1 | Example 2 |
|---|---|---|
| Pesticidal | 50% pesticidal minicell A | 40% pesticidal minicell B |
| minicell | (Photorhabdus derived) | (Bacillus derived) |
| Carrier | 43% kaolin clav | 55% fuller's earth |
| Wetting agent | 2% alkylaryl sulphonate | 2% alkylaryl sulphonate |
| Dispersing | 1% polyethoxylated alcohol | 1% polyethoxylated alcohol |
| agent | | |
| Inert | 4% silica | 2% silica |

Those of skill in the art would also be able to produce water dispersible granules (WDGs) using the teachings contained herein.

### Example 14: Pesticidal Activity of Pesticidal Composition Created from a Wettable Powder

### Materials and Methods

### Pesticidal Composition

A pesticidal composition is created using the wettable powder of Example 13.

Insecticidal Activity Based on Treatment with the Pesticidal Composition Using In Vitro and Artificial Diet Assays

Activity is done as described in Example 3 and Example 7.

### Example 15: Creation of a Suspension Concentration (SC) Pesticidal Composition

### Materials and Methods

### Creation of a Suspension Concentrate Formulation (Minicell Friendly Surfactant to Prevent Caking at High Concentrations)

A minicell as produced in previous examples may be used to produce a suspension concentrate (SC) according to the disclosure. Suspension concentrates are used herein include aqueous formulations in which finely divided solid particles of the pesticidal minicell are stably suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from about 0.5% to about 95% of the concentrate.

### Results

An exemplary suspension concentrate is described in Table 4, below.

TABLE 4
Exemplary suspension concentrate.

| Ingredient | Amount (% w/v) |
|---|---|
| Pesticidal minicell | 40 |
| Naphthalene sulfonate | 4 |
| condensate | |
| Nonionic polymeric | 1 |
| aqueous dispersant | |
| Xanthan gum | 0.5 |
| Preservative | 0.1 |
| Water | Balance |

### Example 16: Seed Treatment and Method of Creating a Plantable Composition

### Materials and Methods

A minicell as produced in previous examples may be used to produce a seed treatment and a plantable composition according to the disclosure. In such seed treatment compositions, in addition to the pesticidal minicell, the compositions may include other pesticides, surfactants, film-forming polymers, carriers, antifreeze agents, and other formulary additives and when used together provide compositions that are storage stable and are suitable for use in normal seed treatment equipment, such as a slurry seed treater, direct treater, on-farm hopper-boxes, planter-boxes, etc.

### Results

An exemplary seed treatment composition is described in Table 5, below.

TABLE 5
Exemplary suspension concentrate.

| Ingredient | Amount |
|---|---|
| Pesticidal minicell | 40% |
| EO/PO Block Co-polymer | 3% |
| Tristyrylphenol ethoxylate | 0.5% |
| Calcium salt, pigment red | 5% |
| Silicone Oil | 0.2% |
| Water | Balance |

A plantable composition may be created by coating a corn seed with the seed treatment composition, thereby creating a novel composition having improved plantability characteristics.

A plantable composition may be created by coating a soybean seed with the seed treatment composition, thereby creating a novel composition having improved plantability characteristics.

A plantable composition may be created by coating a canola seed with the seed treatment composition, thereby creating a novel composition having improved plantability characteristics.

A plantable composition may be created by coating a rice seed with the seed treatment composition, thereby creating a novel composition having improved plantability characteristics.

A plantable composition may be created by coating a wheat seed with the seed treatment composition, thereby creating a novel composition having improved plantability characteristics.

### Example 17: Production of Pesticidal Minicells Further Including an Exogenous Pesticidal Protein

### Materials and Methods

Load with Pesticidal Protein or Expression Cassette (or Alternatively Load Parent with Pesticidal Protein or Expression Cassette)

Creation of P. luminescens minicells is as described in Example 1. Additionally, the gene sequence for a pesticidal protein, by example, Cry1Ac toxin from Bt, is cloned into an expression vector with a CloDF or pMB1 origin of replication behind an inducible promoter, Ptac or Ptet, or constitutive promoter. Alternatively, the pesticidal gene is inserted onto the Photorhabdus luminescens chromosome via homologous recombination using the pPINT vector. Expression of the pesticidal protein is induced with aTc or IPTG at an OD600=0.5 to produce and load the protein within the minicells. Isolation and characterization is as described in previous examples.

### Example 18: Production of Pesticidal Minicells Further Including an Exogenous Pesticidal Nucleic Acid

### Materials and Methods

Load with Pesticidal Nucleic Acid or Expression Cassette for Alternatively Load Parent with Pesticidal Nucleic Acid or Expression Cassette)

Creation of P. luminescens minicells is as described in Example 1. Additionally, to enable stable production of dsRNA, the me gene encoding RNase II needs to be disrupted. The same method for genomic alterations in Photorhabdus as stated in Example 1 would be used to accomplish this task. Another alteration necessary is to add a copy of T7-RNAP to the chromosome using a similar method as stated in Example 1. This allows the use of a T7 promoter-based expression plasmid system. Expression of the dsRNA against Actine is induced by the addition of IPTG. Bioassays are executed as described in previous examples.

### Example 19: Seed Treatment and Method of Creating a Plantable Composition

### Materials and Methods

### UV Stability Assays of Pesticidal Minicells Produced in Example 17 and Example 18

A UV exposure incubator is set up by installing 4 T5 PowerVeg® FS+UV bulbs (EYE Hortilux) in an incubator (Caron) set at 25 C without humidity control. The UV (A+B) irradiation was measured as 1300 mW/cm² on a sample station which is 15 cm under the bulbs. When the lysate or the intact pesticidal minicells with a pesticidal active from E12 and E13 (100 ml) is pipetted in 1.5 ml tube which is sealed by a single layer of Saran wrap (polyethylene) and a rubber band. The tubes are then placed in a rack on the sample station. One set of samples are exposed to UV for 6 hr, 12 hr and 24 hr. The other set of same samples are wrapped in foil and are kept on the sample station in the incubator for the same time intervals.

The UV exposed lysate or the intact pesticidal minicells with a pesticidal active from Example 17 are subjected to artificial diet assays with DBM set up as in Example 3.

1: A pesticidal composition comprising:
a liquid carrier phase; and
a plurality of pesticidal minicells dispersed in the carrier phase,
wherein the plurality of pesticidal minicells are derived from a plurality of a pesticidal parent bacterium comprising at least one genetic mutation causing a modification in a cell partitioning function of the parent bacterium, and
wherein the plurality of pesticidal minicells are present at a particle concentration sufficient to control at least one pest in or on a plant when the composition is applied to the plant.

2: The pesticidal composition of claim 1, wherein at least a portion of the plurality of pesticidal minicells further comprise at least one of: an exogenous pesticidal protein toxin, an exogenous pesticidal nucleic acid, and an exogenous pesticidal active ingredient.

3: The pesticidal composition of claim 2, wherein the portion of the plurality of pesticidal minicells further comprise an exogenous expression cassette coded to express either or both of the exogenous pesticidal protein toxin and the exogenous pesticidal nucleic acid, wherein the exogenous pesticidal protein toxin comprises at least one of a Pir toxin and a Cry toxin, and wherein the exogenous pesticidal nucleic acid is a double-stranded RNA (dsRNA) or a hairpin RNA (hpRNA).

4: The pesticidal composition of claim 2, wherein the exogenous pesticidal active ingredient is selected from the group consisting of an ingredient with fungicidal activity, an ingredient with insecticidal activity, an ingredient with nematocidal activity, an ingredient with selective herbicidal activity, an ingredient with bactericidal activity, and an ingredient with broad spectrum activity.

5: The pesticidal composition of claim 1, wherein the modification in the cell partitioning function of the parent bacterium includes a modification in at least one of a z-ring inhibition protein, a cell division topological specificity factor, or a septum machinery component; wherein the z-ring inhibition protein is selected from the group consisting of a minC polypeptide, a minD polypeptide, and a minE polypeptide; wherein the cell division topological specificity factor is selected from the group consisting of a minE polypeptide and a DivIVA polypeptide; and wherein the septum machinery component is selected from the group consisting of a ftsZ polypeptide and a ftsA polypeptide.

6: The pesticidal composition of claim 1, wherein the pesticidal parent bacterium is selected from the group consisting of Streptomyces avermitilis, Saccharopolyspora spinose, Bacillus thuringiensis, Brevibacillus laterosporus, Clostridium bifermentans, Bacillus popilliae, Bacillus subtilis, Bacillus amyloliquefaciens, Photorhabdus luminescens, Xenorhabdus nematophila, Serratia entomophila, Yersinia entomophaga, Pseudomonas entomophila, Burkholderia spp., Chromobacterium subtsugae, and Escherichia coli.

7: The pesticidal composition of claim 6, wherein the pesticidal parent bacterium is selected from the group consisting of Bacillus subtilis strain RTI477, Bacillus subtilis strain ATCC 6633, Bacillus subtilis strain ATCC 21332, Bacillus subtilis strain 168, Bacillus subtilis strain ATCC 9943, Bacillus subtilis strain QST713, and Bacillus subtilis strain NCIB 3610, Bacillus atrophaeus strain ABI02A DSM 32019, Bacillus atrophaeus strain ABI03 DSM 32285, Bacillus atrophaeus strain ABI05 DSM 24918, Bacillus amyloliquefaciens strain RTI301, Bacillus amyloliquefaciens FZB24, Bacillus amyloliquefaciens FZB42, Bacillus amyloliquefaciens BA-1, Bacillus amyloliquefaciens IMG 5-29032, Bacillus amyloliquefaciens MBI600, Bacillus amyloliquefaciens CECT8836, and Bacillus amyloliquefaciens M4 (S499).

8: The pesticidal composition of claim 6, wherein the pesticidal parent bacterium is Photorhabdus luminescens, and wherein the pesticidal minicell comprises the exogenous pesticidal protein toxin Pir.

9: The pesticidal composition of claim 6, wherein the pesticidal parent bacterium is Bacillus subtilis, and wherein the pesticidal minicell comprises the exogenous pesticidal molecule.

10: The pesticidal composition of claim 6, wherein the pesticidal parent bacterium is a genetically modified Escherichia coli expressing one or more exogenous pesticidal active ingredients.

11: The pesticidal composition of claim 1, wherein the composition is applied to the plant as at least one of a foliar treatment, an injection treatment, a pre-emergence treatment, and a post-emergence treatment.

12: The pesticidal composition of claim 1, wherein the composition is formulated as at least one of a Ready To Use (RTU) formulation, a suspension concentrate, a tank-mix, an aerosol, a seed treatment, a root dip, a soil treatment, an irrigation formulation, a sprinkler formulation, and a drench treatment.

13: The pesticidal composition of claim 1, further comprising agrochemical surfactants, wherein the agrochemical surfactants improve at least one of the characteristics of sprayability, spreadability, and injectability.

14: The pesticidal composition of claim 1, wherein the liquid carrier phase is aqueous or oil.

15: The pesticidal composition of claim 2, further comprising at least one of: an exogenous pesticidal protein toxin, an exogenous pesticidal nucleic acid, and an exogenous pesticidal active ingredient dispersed in the carrier phase, wherein the exogenous pesticidal protein toxin comprises a Pir toxin and a Cry toxin, and wherein the exogenous pesticidal nucleic acid is a double-stranded RNA (dsRNA) or precursor thereof, a hairpin RNA (hpRNA) or precursor thereof, or a microRNA (miRNA) or precursor thereof.

16: The pesticidal composition of claim 15, wherein the exogenous pesticidal active ingredient is selected from the group consisting of an ingredient with fungicidal activity, an ingredient with insecticidal activity, an ingredient with nematocidal activity, an ingredient with selective herbicidal activity, an ingredient with bactericidal activity, and an ingredient with broad spectrum activity.

17: The pesticidal composition of claim 2, wherein the pesticidal activity of the minicell and the pesticidal activity of the exogenous pesticidal protein toxin, the exogenous pesticidal nucleic acid, or the exogenous pesticidal active ingredient target the same pest, or wherein the pesticidal activity of the minicell and the pesticidal activity of the exogenous pesticidal protein toxin, the exogenous pesticidal nucleic acid, or the exogenous pesticidal active ingredient target different pests.

18: The pesticidal composition of claim 15, wherein the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target the same pest, or wherein the pesticidal activity of the exogenous pesticidal protein toxin, the exogenous pesticidal nucleic acid, or the exogenous pesticidal active ingredient and the pesticidal activity of the exogenous pesticidal protein toxin, the exogenous pesticidal nucleic acid, or the exogenous pesticidal active ingredient dispersed in the carrier phase target different pests.

19: A method of making pesticidal minicells, comprising the steps of:
a) providing a pesticidal parent bacterium comprising at least one genetic mutation causing a modification in a cell partitioning function of the parent bacterium;
b) growing the pesticidal parent bacterium under conditions allowing the formation of pesticidal minicells; and
c) purifying pesticidal minicells using centrifugation, tangential flow filtration (TFF), or TFF and centrifugation.

20: A method of controlling a pest, the method comprising:
applying the pesticidal composition of claim 1 to a plant or an area to be planted.


SEQUENCE LISTING (6 sequences)

SEQ ID NO: 1 - DNA, 24 bp, Artificial Sequence
  Synthetic Construct
tggtaatctatgtatcctggcaac

SEQ ID NO: 2 - DNA, 20 bp, Artificial Sequence
  Synthetic Construct
ttcgccagatgataaggaac

SEQ ID NO: 3 - DNA, 35 bp, Artificial Sequence
  Synthetic Construct
ggtctcggcattctcgcaatattatccatcctgcc

SEQ ID NO: 4 - DNA, 33 bp, Artificial Sequence
  Synthetic Construct
taggtctcgtctcgatataaaggcacaaagcgg

SEQ ID NO: 5 - DNA, 21 bp, Artificial Sequence
  Synthetic Construct
atacttgtccactttgcaccg

SEQ ID NO: 6 - DNA, 20 bp, Artificial Sequence
  Synthetic Construct
ttcgggtagacaaattgcac